(19) United States
(12) Patent Application Publication
(10) Pub. No.: US 2022/0168361 A1
(43) Pub. Date: Jun. 2, 2022

(54) METHODS FOR MEASURING THERAPEUTIC EFFECTS OF RETINAL DISEASE THERAPIES

(71) Applicant: Lineage Cell Therapeutics, Inc., Carlsbad, CA (US)

(72) Inventors: Oscar Cuzzani, Walnut Creek, CA (US); Francois Binette, Napa, CA (US); Gary Hogge, San Francisco, CA (US)

(21) Appl. No.: 17/547,133

(22) Filed: Dec. 9, 2021

Related U.S. Application Data
(62) Division of application No. 16/494,498, filed on Sep. 16, 2019.
(60) Provisional application No. 62/585,520, filed on Nov. 13, 2017.
(60) Provisional application No. 62/501,690, filed on May 4, 2017.
(60) Provisional application No. 62/472,544, filed on Mar. 16, 2017.

(51) Int. Cl. A61K 35/545; A61P 27/02

(52) U.S. Cl. CPC A61K 35/545; A61P 27/02

(57) ABSTRACT

Described herein are compositions and methods for treating retinal diseases or disorders using RPE cells.

### CROSS-REFERENCE TO RELATED APPLICATIONS

This application claims priority to, and the benefit of, U.S. provisional patent application Ser. No. 62/472,544 filed on Mar. 16, 2017, U.S. provisional patent application Ser. No. 62/501,690 filed on May 4, 2017, and U.S. provisional patent application Ser. No. 62/585,520 filed on Nov. 13, 2017, each of which are incorporated herein by reference in their entirety.

### BACKGROUND

The present disclosure pertains generally to the field of treating retinal diseases, and more particularly to treating retinal diseases using human embryonic stem cell derived retinal pigment epithelial (RPE) cell compositions.

Dysfunction, degeneration and loss of RPE cells are prominent features of retinal diseases such as AMD, Best Disease and subtypes of Retinitis Pigmentosa (RP). AMD is the leading cause of visual disability in the Western world. Among people over 75 years of age, 25-30% are affected by Age-Related Macular Degeneration (AMD), with progressive central visual loss that leads to blindness in 6-8% of the patients. The retinal degeneration primarily involves the macula, the central part of the retina responsible for fine visual detail and color perception facial recognition, reading and driving. The dry form of AMD is initiated by hyperplasia of the RPE and formation of drusen deposits underneath the RPE or within the Bruch's membrane consisting of metabolic end products. The disease may gradually progress into the advanced stage of geographic atrophy (GA) with degeneration of RPE cells and photoreceptors over large areas of the macula, causing central visual loss.

The pathogenesis of the disease involves abnormalities in four functionally interrelated tissues, i.e., retinal pigment epithelium (RPE), Bruch's membrane, choriocapillaries and photoreceptors. However, impairment of RPE cell function is an early and crucial event in the molecular pathways leading to clinically relevant AMD changes.

There is currently no effective or approved treatment for dry-AMD. Prophylactic measures include vitamin/mineral supplements. These reduce the risk of developing wet AMD but do not affect the development of progression of geographic atrophy.

In cases where the center of the fovea is not affected, the best-corrected visual acuity (BCVA) score may not be affected either, because BCVA is a measure of the central acuity of the fovea. Although, BCVA is widely accepted by the clinical community and regulatory authorities worldwide as a key measure of visual function and represents the gold standard by which the efficacy of treatment of retinal disease is judged, it can sometimes fail to assess nuances of comprehensive visual function. It has been demonstrated that in subjects with BCVA of 20/50 or better, other features of visual function can be significantly impaired, including contrast sensitivity, low-luminance BCVA, and reading speed. In addition, best-corrected visual acuity alone cannot sufficiently measure the progression of visual deficits in all subjects, including those with foveal-sparing GA.

### SUMMARY

Retinal pigment epithelium (RPE) cells and RPE cell compositions have been developed that are useful for the treatment of retinal diseases and disorders, including preventing the progression of retinal degeneration and vision loss. When administered to a subject in need, these RPE cells and cell compositions safely promote the engraftment, integration, survival and function of the ocular structure.

Impairment of visual function, retinal disease progression and the effects of retinal disease treatments can be detected and monitored using technologies that assess quantitative morphology, even in subjects with nonimpaired BCVA. Clinical studies involving subjects with AMD and GA that aim to quantify changes in visual function and correlate them with disease progression can incorporate additional assessments that account for the underlying pathophysiologic processes of the disease. Also disclosed herein are methods for measuring the therapeutic effects of retinal disease therapies using improved quantitative structural and functional assessments.

Provided herein according to some aspects are methods of treating or slowing the progression of a retinal disease or disorder, the method comprising, administering a therapeutically effective amount of a pharmaceutical composition comprising retinal pigment epithelium (RPE) cells to a subject.

In some embodiments, the administering of the therapeutically effective amount of retinal pigment epithelium (RPE) cells results in a best corrected visual acuity (BCVA) that does not decrease as measured from a baseline for about 1 day to about 3 months, 1 day to about 15 months or from 1 day to about 24 months or from about 90 days to about 24 months.

In some embodiments, the subject comprises a BCVA of 20/64 or less; 20/70 or less; or from between about 20/64 and about 20/400.

In some embodiments, the administering of the therapeutically effective amount of retinal pigment epithelium (RPE) cells results in a best corrected visual acuity (BCVA) that remains stable as measured from a baseline for about 1 day to about 15 months, or from 1 day to about 24 months or from about 90 days to about 24 months.

In some embodiments, the administering of the therapeutically effective amount of retinal pigment epithelium (RPE) cells results in about 89% to about 96% of subjects having an increase in pigmentation. In other embodiments, the increase in pigmentation remains for at least about 6 months to about 12 months, or from about 90 days to about 24 months. In still other embodiments, the administering of the therapeutically effective amount of retinal pigment epithelium (RPE) cells results in retinal pigmentation.

In further embodiments, the administering of the therapeutically effective amount of retinal pigment epithelium (RPE) cells results in an increase in retinal pigmentation as measured from a baseline for at least about 2 months to about 1 year, or from 90 days to about 24 months. In other embodiments, in about 2 to about 12 months after administration, retinal pigmentation is stabilized or from about 90 days to about 24 months. In yet another embodiment, about 3 to about 9 months after administration, the retinal pigmentation is stabilized.

According to some aspects of the present disclosure, the subretinal fluid within a bleb in which the cells are administered is absorbed within less than 48 hours.

According to other aspects, the administering of the therapeutically effective amount of retinal pigment epithelium (RPE) cells results in recovery of an ellipsoid zone. In yet other aspects, recovery of an ellipsoid zone comprises recovery according to an ellipsoid zone analysis.

In some embodiments, an ellipsoid zone analysis comprises a visual analysis of the ellipsoid zone, wherein the ellipsoid zone of a subject is compared to age-matched, sex-matched control, a baseline or a fellow eye.

According to further embodiments, recovery is indicated by restoration of normal architecture as compared to age-matched, sex-matched control, a baseline or a fellow eye. According to other embodiments, recovery comprises the subjective assessment that one or more of the following are becoming more organized, including the, external limiting membrane, myoid zone (inner segments of photoreceptors), ellipsoid zone (IS/OS Junction), outer segments of the photoreceptors, loss of drusen, and disappearance of reticular pseudo-drusen. In some embodiments, recovery comprises the subjective assessment that one or more of the basic foundational layers of the retina are becoming more organized.

According to certain embodiments, the basic foundational layers of the retina becoming more organized comprise one or more of the external limiting membrane, myoid zone (inner segments of photoreceptors), ellipsoid zone (IS/OS Junction), and outer segments of the photoreceptors.

According to other embodiments, new or worsening ERMs do not require surgical removal within from about 1 week to about 12 months of administration, or from about 1 week to about 24 months, or from about 90 days to about 24 months.

According to some embodiments, the RPE cells do not show tumorigenicity within about 1 week to about 1 year of administration, or from about 1 week to about 24 months, or from about 90 days to about 24 months.

According to some embodiments, the RPE cells show from 0% to about 5% histologic tumorigenicity within about 9 months of administration.

According to some embodiments, the administering of the therapeutically effective amount of retinal pigment epithelium (RPE) cells does not result in retinal breaks or ruptures.

According to some embodiments, the administering of the therapeutically effective amount of retinal pigment epithelium (RPE) cells does not result in retinal edema.

According to some embodiments, the therapeutically effective amount of RPE cells is between about 50,000 and 5,000,000 cells per administration.

According to some embodiments, the therapeutically effective amount of RPE cells is about 200,000 cells per administration.

According to some embodiments, the therapeutically effective amount of RPE cells is about 500,000 cells per administration.

According to some embodiments, the pharmaceutical composition comprises about 500 cells per μ1 to about 10,000 cells per μl.

According to some embodiments, when said amount is 50,000 cells per administration, the pharmaceutical composition comprises about 500-1,000 cells per μl.

According to some embodiments, when said amount is 200,000 cells per administration, the pharmaceutical composition comprises about 2,000 cells per μl.

According to some embodiments, when said amount is 500,000 cells per administration, the pharmaceutical composition comprises about 5,000 cells per μl.

According to some embodiments, when said amount is 1,000,000 cells per administration, the pharmaceutical composition comprises about 10,000 cells per μl.

According to some embodiments, at least 95% of the cells co-express premelanosome protein (PMEL17) and cellular retinaldehyde binding protein (CRALBP).

According to some embodiments, the trans-epithelial electrical resistance of the cells is greater than 100 ohms to the subject.

According to some embodiments, the RPE cells are generated by ex-vivo differentiation of human embryonic stem cells.

According to some embodiments, administering comprises: implanting RPE cells.

According to some embodiments, the methods described herein further comprise, prior to RPE cell implantation, preparation of the RPE dose. According to some embodiments, preparation of the dose of RPE comprises thawing the dose. According to some embodiments, preparation of the dose of RPE comprises mixing the RPE cells and loading into the delivery device.

According to some embodiments, the methods described herein further comprise, prior to RPE cell implantation, performing a vitrectomy. According to some embodiments, performing a vitrectomy comprises administering triamcinolone to stain the vitreous and removal of vitreous traction.

According to certain embodiments, the methods described herein further comprise, prior to performing a vitrectomy, cleaning the surgical site.

According to some embodiments, the methods described herein further comprise, after implanting RPE cells, cleaning the surgical site.

According to some embodiments, administering comprises: cleaning the surgical site, performing a vitrectomy, preparation of the RPE dose, and RPE cell implantation.

According to some embodiments, implanting RPE cells comprises injecting the RPE cells at least 1-disc diameter away from the edge of the geographic atrophy (GA) lesion.

According to some embodiments, implanting RPE cells comprises injecting the RPE cells in one or more of the following: covering a GA lesion, covering the fovea, covering portions or all of the transitional zone bordering the GA lesion, or covering surrounding healthy tissue adjacent to a GA lesion.

According to some embodiments, the transitional zone comprises an area between intact and degenerating retina.

According to some embodiments, covering a GA lesion comprises coving the entire GA lesion with a bleb. According to other embodiments, the GA size comprises from 0.1 mm² to about 50 mm²; from about 0.5 mm² to about 30 mm²; from about 0.5 mm² to about 15 mm²; from about 0.1 mm² to about 10 mm²; from about 0.25 mm² to about 5 mm² or any point between two points.

According to some embodiments, administering comprises: administering RPE cells such that the central macular vision is preserved.

According to some embodiments, the RPE cells are generated by: (a) culturing human embryonic stem cells or induced pluripotent stem cells in a medium comprising nicotinamide so as to generate differentiating cells; (b) culturing said differentiating cells in a medium comprising nicotinamide and acitivin A to generate cells which are further differentiated towards the RPE lineage; and (c) culturing said cells which are further differentiated towards the RPE lineage in a medium comprising nicotinamide, wherein said medium is devoid of activin A.

According to some embodiments, the embryonic stem cells or induced pluripotent stem cells are propagated in a medium comprising bFGF and TGFI3 under non-adherent conditions. According to further embodiments, the medium of (a) is substantially is devoid of activin A.

According to some embodiments, the cells are administered in a single administration. According to some embodiments, the cells are administered into the subretinal space of the subject. According to some embodiments, subretinal administration is transvitreal or suprachoroidal. According to some embodiments, administration is by cannula.

According to some embodiments, the healing of the site of administration by the cannula is within about 1 day to about 30 days. According to some embodiments, the healing of the site of administration by the cannula is within about 5 days to about 21 days or within about 7 days to about 15 days.

According to some embodiments, the methods described herein further comprise, administering immunosuppression to the subject for one day to three months after the administration of RPE cells.

According to other embodiments, the methods described herein further comprise, administering immunosuppression to the subject for three months after the administration of RPE cells.

According to yet other embodiments, the methods described herein further comprise, administering immunosuppression to the subject for one day to one month after the administration of RPE cells.

According to some embodiments, the retinal disease or condition is selected from the group consisting of intermediate dry AMD, retinitis pigmentosa, retinal detachment, retinal dysplasia, retinal atrophy, retinopathy, macular dystrophy, cone dystrophy, cone-rod dystrophy, Malattia Leventinese, Doyne honeycomb dystrophy, Sorsby's dystrophy, pattern/butterfly dystrophies, Best vitelliform dystrophy, North Carolina dystrophy, central areolar choroidal dystrophy, angioid streaks, toxic maculopathy, Stargardt disease, pathologic myopia, retinitis pigmentosa, and macular degeneration.

According to some embodiments, the disease is age-related macular degeneration. According to some embodiments, said age-related macular degeneration is dry-form age-related macular degeneration.

Provided herein, according to some aspects are methods of increasing the safety of a method of treating a subject with dry AMD, comprising, administering a therapeutically effective amount of retinal pigment epithelium (RPE) cells to a subject, wherein the subject is not administered systemic immunosuppression.

According to some embodiments, the incidence and frequency of treatment emergent adverse events is lower than with immunosuppression.

Provided herein, according to some aspects are methods of organizing the ellipsoid zone of the retina in a subject with GA, comprising: administering of the therapeutically effective amount of retinal pigment epithelium (RPE) cells, wherein after administration a disorganized ellipsoid zone becomes organized.

According to some embodiments, recovery of an ellipsoid zone comprises recovery according to an ellipsoid zone analysis.

According to some embodiments, an ellipsoid zone analysis comprises a visual analysis of the ellipsoid zone, wherein the ellipsoid zone of a subject is compared to age-matched, sex-matched control, a baseline, or a fellow eye.

According to some embodiments, recovery is indicated by restoration of normal architecture as compared to age-matched, sex-matched control, a baseline, or a fellow eye.

According to some embodiments, recovery comprises the subjective assessment that one or more of the following are becoming more organized, including the, external limiting membrane, myoid zone (inner segments of photoreceptors), ellipsoid zone (IS/OS Junction), outer segments of the photoreceptors, loss of drusen, and disappearance of reticular pseudo-drusen.

According to some embodiments, recovery comprises the subjective assessment that one or more of the basic foundational layers of the retina are becoming more organized.

According to some embodiments, the basic foundational layers of the retina becoming more organized comprise one or more of the external limiting membrane, myoid zone (inner segments of photoreceptors), ellipsoid zone (IS/OS Junction), and outer segments of the photoreceptors.

According to some embodiments, the subject comprises a BCVA of 20/64 or less; 20/70 or less;

or from between about 20/64 and about 20/400.

According to some embodiments, treating or slowing the progression of a retinal disease is demonstrated by microperimetry assessed recovery of vision, wherein microperimetry assessed recovery of vision comprises a correlation between retinal sensitivity on microperimetry and EZ defect as compared to a baseline.

According to other embodiments, microperimetry assessed recovery of vision comprises demonstrating that sites of the retina near or at the site of administration of the RPE cells comprises an improved microperimetry assessment compared to a baseline microperimetry assessment.

According to certain embodiments, treating or slowing the progression of a retinal disease comprises a reduction in rate of GA lesion growth relative to a baseline or fellow eye of between about 5% and about 20% at one year after administration; or between about 5% and about 50%; or between about 5% and about 25%; or between about 5% and about 100%; between about 5% and about 10%.

According to some embodiments, treating or slowing the progression of a retinal disease comprises one or more of: a stable BCVA; no deterioration in low luminance test performance; or no deterioration in microperimetry sensitivity; or no deterioration in reading speed, when compared to age-matched, sex-matched control, a baseline, or a fellow eye, wherein the comparison is at one or more of, one month, at three months, at six months or at one year.

According to some embodiments, a pharmaceutical composition for treating or slowing the progression of a retinal disease or disorder comprising as an active substance about between 50,000 and 500,000 RPE cells is presented.

According to other embodiments, a pharmaceutical composition for stabilizing the RPE of a subject with a retinal disease or disorder comprising as an active substance about between 50,000 and 500,000 RPE cells is presented.

According to some embodiments, the RPE cells are characterized by the following features:

(a) at least 95% of the cells co-express premelanosome protein (PMEL17) and cellular retinaldehyde binding protein (CRALBP); and
(b) the trans-epithelial electrical resistance of the cells is greater than 100 ohms to a subject in which the cells were administered; wherein from about 90 days to about 24 months after administration, retinal pigmentation in the subject is stabilized.

According to some embodiments, recovery of an ellipsoid zone comprises improvement in one or more of, EZ-RPE thickness, area, or volume measurements.

According to some embodiments, improvement in one or more of EZ-RPE thickness, area, or volume measurements is inversely correlated with visual acuity.

According to some embodiments, the ellipsoid zone analysis demonstrates organization of the EZ by a decrease in the EZ volume as compared to an age-matched, sex-matched control, a baseline or a fellow eye.

According to some embodiments, the decrease in the EZ volume comprises at least 2% or at least 5% or at least 7% or at least 10%, or between 1 and 5% or between 1 and 10% or between 1 and 50% or between 10 and 50%.

According to some embodiments, organization of the EZ comprises a decrease in volume of the structures of the EZ from a baseline by at least 2%, by at least 5%, by at least 10%, by between about 1% and about 50%.

According to some embodiments, the treating or slowing the progression of a retinal disease or disorder is enhanced by the cells secretion of tropic factors.

### BRIEF DESCRIPTION OF THE DRAWINGS

The technology described herein will be more fully understood by reference to the following drawings, which are for illustrative purposes only:

FIG. 1 is an illustration of cell-based therapy to replace and support dysfunctional and degenerated RPE in dry AMD with GA.

FIG. 2A is a graph of the best corrected visual acuity (BCVA) measured over 1 year for the treated eyes of cohort 1 (patients 1, 2, and 3 (Pt. 1, Pt. 2. Pt. 3)), treated with a dose of about 50,000 RPE cells.

FIG. 2B is a graph of the best corrected visual acuity (BCVA) measured over 1 year for the fellow eyes of cohort 1 (patients 1, 2, and 3 (Pt. 1, Pt. 2. Pt. 3)).

FIGS. 2C-2F The BCVA remained stable and did not decrease in the treated eyes of cohort 2 (patients 4, 5, and 6) and was stable in fellow eyes, as shown in FIG. 2C, FIG. 2D, FIG. 2E, and FIG. 2F. Individual patients' treated eyes are shown in FIG. 2C and FIG. 2E.

FIG. 3 shows color fundus images for cohort 1 (patients 1, 2, and 3 (Pt. 1, Pt. 2. Pt. 3)) at pre-operation (pre-op) and during surgery (intra-op) time points.

FIG. 4 shows color fundus imaging for cohort 1 (patients 1, 2, and 3 (Pt. 1, Pt. 2. Pt. 3) prior to treatment with a target does of 50,000 RPE cells (pre-op) and 2-months after treatment.

FIG. 5 shows color fundus imaging for cohort 1 (patients 1, 2, and 3 (Pt. 1, Pt. 2. Pt. 3) prior to treatment with a target does of 50,000 RPE cells (pre-op) and 9-months to 1 year after treatment (post-op) time points.

FIG. 6 shows blue auto fluorescence images from patient 1 (cohort 1, treated with a dose of 50,000 RPE cells) at pre-op, 1-day, 1-week, 2-month, 4.5-month, and 9-month post-op time points.

FIG. 7 shows blue auto fluorescence images from patient 2 at pre-op, 1-day, 1-week, 2-month, 6-month, and 9-month post-op time points.

FIG. 8 shows blue auto fluorescence images from patient 3 at pre-op, 1-day, 1-week, 2-month, 7-month, and 9-month post-op time points.

FIG. 9 shows a color image at the time of surgery (day 0), FAF and color images at day-1 post op, and color images at 2-months, 3-months, 4-months and 6-months post-op for patient 4 of cohort 2 (200,000 RPE cell suspension dose).

FIG. 10 shows color and corresponding FAF images for patient 5 of cohort 2 (200,000 RPE cell suspension dose) at day 0, month 1, month 2, month 3, and month 6.

FIG. 11 shows OCT images of the healing injection site for cohort 1.

FIG. 12 shows OCT scans for patient 1 at pre-op, 1-week, 1-month, and 1-year post-op time points.

FIG. 13 shows OCT scans for patient 2 at pre-op, 1-month and 9-month post-op time points.

FIG. 14 shows OCT scans for patient 3 at pre-op, 3-month and 9-month post-op time points.

FIG. 15 shows OCT and infrared OCT scans for patient 4 of cohort 2 (200,000 RPE cell suspension dose) at pre-op, 1-month and 9-month post-op time points.

FIG. 16 shows OCT scans for patient 5 of cohort 2 (200,000 RPE cell suspension dose) at baseline, 1-week, 2-weeks, 1-month, 2-month, 3 month and 6-month post-op time points.

FIG. 17 shows OCT scans, an infrared image, and histological images after subretinal transplantation of hESC-RPE cells in porcine eyes.

FIG. 18 shows a benign teratoma in the subretinal space of a NOD-SKID mouse.

FIG. 19 shows hESC-derived RPE cells in the subretinal space of a NOD-SKID mouse treated with 100,000 hESC-derived RPE cells in solution.

FIG. 20 shows Hunt+ cells in the subretinal space of a NOD-SKID mouse treated with 100,000 hESC-derived RPE cells in solution.

FIG. 21 shows the engraftment and survival of hESC-derived RPE in three animal species using stains that indicate the presence of human cells.

FIG. 22A shows a blue auto fluorescence image from patient 8 (cohort 3; dose of 100,000 RPE cells/50 μL) taken before surgery, showing a baseline image of the GA (dark area), the outline of the future bleb border (dotted line) and the precise implantation location (star).

FIG. 22B shows a color fundus image from patient 8 taken before surgery, showing a baseline image of the GA (dark area), the outline of the future bleb border (dotted line) and the precise implantation location (star).

FIG. 22C shows a color image taken of the bleb implanted at the time of surgery.

FIG. 23 shows a color fundus image at 1 month for patient 8.

FIG. 24A shows a blue auto fluorescence image taken at 1 month for patient 8.

FIG. 24B shows a blue auto fluorescence image taken at 2 months for patient 8.

FIG. 24C shows a blue auto fluorescence image taken at 3 months for patient 8.

FIG. 25 shows infrared and corresponding OCT images of the transition zone at time points of baseline (prior to surgery), 1 month, 2 months and 3 months for patient 8.

FIG. 26 shows infrared and corresponding OCT images of the transition zone at time points of baseline (prior to surgery), 1 month, 2 months and 3 months for patient 8.

FIG. 27 shows infrared and corresponding OCT images of the transition zone at time points of baseline (prior to surgery), 1 month, 2 months and 3 months for patient 8.

### DETAILED DESCRIPTION

RPE cell compositions and methods described herein may be used in slowing the progression of retinal degenerative diseases or disorders, slowing the progression of age related macular degeneration (AMD) or intermediate age related macular degeneration (AMD) preventing retinal degenerative disease, preventing AMD, restoring retinal pigment epithelium (RPE), increasing RPE, replacing RPE or treating RPE diseases, defects, conditions and/or injuries in a subject by administering to the subject a composition comprising the RPE cells. For example, human embryonic stem cell derived RPE cell compositions can be injected into the subretinal space to promote restoration of the RPE and to prevent the progression of retinal degradation caused by a retinal disease or condition.

In certain embodiments, RPE cells are administered over a GA lesion or over surrounding healthy tissue near a GA lesion. Administering over the GA lesion will assist in repairing or correction the lesion. Administering of RPE cells over surrounding healthy tissue near a GA lesion will prevent further growth of the lesion.

In certain embodiments, RPE cell implants provide long-lasting trophic support to degenerating retinal tissue by secreting these factors once implanted. This tropic support may act to attenuate retinal degradation and vision loss is some subjects. Trophic factors are known as cell survival and differentiation-promoting agents. Examples of trophic factors and tropic factor families include but are limited to, neurotrophins, the ciliary neurotrophic factor/leukemia inhibitory factor (CNTF/LIF) family, hepatocyte growth factor/scatter factor family, insulin-like growth factor (IGF) family, and the glial cell line-derived neurotrophic factor (GDNF) family. The RPE cells described herein may start secreting trophic factors immediately after administration or retinal grafting. In addition, a steady stream of neuroprotective support may start when the cells integrate in between the recipient cells and establish synaptic contacts with the subject's cells.

In certain embodiments, the retinal degenerative disease may be one or more of: RPE dysfunction, photoreceptor dysfunction, accumulation of lipofuscin, formation of drusen, or inflammation.

In other embodiments, the retinal degenerative disease is selected from at least one of retinitis pigmentosa, lebers congenital amaurosis, hereditary or acquired macular degeneration, age related macular degeneration (AMD), Best disease, retinal detachment, gyrate atrophy, choroideremia, pattern dystrophy, RPE dystrophies, Stargardt disease, RPE and retinal damage caused by any one of photic, laser, infection, radiation, neovascular or traumatic injury. In yet other embodiments, the AMD is geographic atrophy (GA).

In certain embodiments, the RPE defects may result from one or more of: advanced age, cigarette smoking, unhealthy body weight, low intake of antioxidants, or cardiovascular disorders. In other embodiments, the RPE defects may result from a congenital abnormality.

“Retinal pigment epithelium cells”, “RPE cells”, “RPEs”, which may be used interchangeably as the context allows, refers to cells of a cell type that is for example, functionally, epi-genetically, or by expression profile similar to that of native RPE cells which form the pigment epithelium cell layer of the retina (e.g., upon transplantation, administration or delivery within an eye, they exhibit functional activities similar to those of native RPE cells).

According to some embodiments, the RPE cell expresses at least one, two, three, four or five markers of mature RPE cells. According to some embodiments, the RPE cell expresses between at least two to at least ten or at least two to at least thirty markers of mature RPE cells. Such markers include, but are not limited to CRALBP, RPE65, PEDF, PMEL17, bestrophin 1 and tyrosinase. Optionally, the RPE cell may also express a marker of a RPE progenitor (e.g., MITF). In other embodiments, the RPE cells express PAX-6. In other embodiments, the RPE cells express at least one marker of a retinal progenitor cell including, but not limited to Rx, OTX2 or SIX3. Optionally, the RPE cells may express either SIX6 and/or LHX2.

As used herein the phrase “markers of mature RPE cells” refers to antigens (e.g., proteins) that are elevated (e.g., at least 2-fold, at least 5-fold, at least 10-fold) in mature RPE cells with respect to non RPE cells or immature RPE cells.

As used herein the phrase “markers of RPE progenitor cells” refers to antigens (e.g., proteins) that are elevated (e.g. at least 2-fold, at least 5-fold, at least 10-fold) in RPE progenitor cells when compared with non RPE cells.

According to other embodiments, the RPE cells have a morphology similar to that of native RPE cells which form the pigment epithelium cell layer of the retina. For example, the cells may be pigmented and have a characteristic polygonal shape.

According to still other embodiments, the RPE cells are capable of treating diseases such as macular degeneration.

According to additional embodiments, the RPE cells fulfill at least 1, 2, 3, 4 or all of the requirements listed herein above.

As used herein, the phrase “stem cells” refers to cells which are capable of remaining in an undifferentiated state (e.g., pluripotent or multipotent stem cells) for extended periods of time in culture until induced to differentiate into other cell types having a particular, specialized function (e.g., fully differentiated cells). Preferably, the phrase “stem cells” encompasses embryonic stem cells (ESCs), induced pluripotent stem cells (iPSCs), adult stem cells, mesenchymal stem cells and hematopoietic stem cells.

According to some embodiments, the RPE cells are generated from pluripotent stem cells (e.g., ESCs or iPSCs).

Induced pluripotent stem cells (iPSCs) can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 [Yamanaka S, Cell Stem Cell. 2007, 1(1):39-49; Aoi T, et al., Generation of Pluripotent Stem Cells from Adult Mouse Liver and Stomach Cells. Science. 2008 Feb. 14. (Epub ahead of print); IH Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell 2007; 131:861-872]. Other embryonic-like stem cells can be generated by nuclear transfer to oocytes, fusion with embryonic stem cells or nuclear transfer into zygotes if the recipient cells are arrested in mitosis. In addition, iPSCs may be generated using non-integrating methods e.g., by using small molecules or RNA.

The phrase “embryonic stem cells” refers to embryonic cells that are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO 2006/040763) and embryonic germ (EG) cells which are obtained from the genital tissue of a fetus any time during gestation, preferably before 10 weeks of gestation. The embryonic stem cells of some embodiments of the present disclosure can be obtained using well-known cell-culture methods. For example, human embryonic stem cells can be isolated from human blastocysts.

Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by a procedure in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells, see Reubinoff et al. Nat Biotechnol 2000, May: 18(5): 559; Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; and Gardner et al., [Fertil. Steril. 69: 84, 1998].

It will be appreciated that commercially available stem cells can also be used according to some embodiments of the present disclosure Human ES cells can be purchased from the NIH human embryonic stem cells registry, www.grants.nih.govstem_cells/or from other hESC registries. Non-limiting examples of commercially available embryonic stem cell lines are HAD-C 102, ESI, BGO 1, BG02, BGO3, BG04, CY12, CY30, CY92, CY1O, TEO3, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WAO 1, UCSF4, NYUES 1, NYUES2, NYUES3, NYUES4, NYUESS, NYUES6, NYUES7, UCLA 1, UCLA 2, UCLA 3, WA077 (H7), WA09 (H9), WA 13 (H13), WA14 (H14), HUES 62, HUES 63, HUES 64, CT I, CT2, CT3, CT4, MA135, Eneavour-2, WIBR 1, WIBR2, WIBR3, WIBR4, WIBRS, HUES 45, Shef 3, Shef 6, BINhem19, BJNhem20, SAGO 1, SAOO1.

According to some embodiments, the embryonic stem cell line is HAD-C102 or ESI.

In addition, ES cells can be obtained from other species, including mouse (Mills and Bradley, 2001), golden hamster [Doetschman et al., 1988, Dev Biol. 127: 224-7], rat [Iannaccone et al., 1994, Dev Biol. 163: 288-92], rabbit [Giles et al. 1993, Mol Reprod Dev. 36: 130-8; Graves & Moreadith, 1993, Mol Reprod Dev. 1993, 30 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci USA. 92: 7844-8; Thomson et al., 1996, Biol Reprod. 55: 254-9].

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, Mo., USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Another method for preparing ES cells is described in Chung et al., Cell Stem Cell, Volume 2, Issue 2, 113-117, 7 Feb. 2008. This method comprises removing a single cell from an embryo during an in vitro fertilization process. The embryo is not destroyed in this process.

EG (embryonic germ) cells are prepared from the primordial germ cells obtained from fetuses of about 8-11 weeks of gestation (in the case of a human fetus) using laboratory techniques known to anyone skilled in the arts. The genital ridges are dissociated and cut into small portions which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

Yet another method for preparing ES cells is by parthenogenesis. The embryo is also not destroyed in the process.

ES culturing methods may include the use of feeder cell layers which secrete factors needed for stem cell proliferation, while at the same time, inhibiting their differentiation. The culturing is typically effected on a solid surface, for example a surface coated with gelatin or vimentin. Exemplary feeder layers include human embryonic fibroblasts, adult fallopian epithelial cells, primary mouse embryonic fibroblasts (PMEF), mouse embryonic fibroblasts (MEF), murine fetal fibroblasts (MFF), human embryonic fibroblast (HEF), human fibroblasts obtained from the differentiation of human embryonic stem cells, human fetal muscle cells (HFM), human fetal skin cells (HFS), human adult skin cells, human foreskin fibroblasts (HFF), human umbilical cord fibroblasts, human cells obtained from the umbilical cord or placenta, and human marrow stromal cells (hMSCs). Growth factors may be added to the medium to maintain the ESCs in an undifferentiated state. Such growth factors include bFGF and/or TGF. In another embodiment, agents may be added to the medium to maintain the hESCs in a naive undifferentiated state—see for example Kalkan et al., 2014, Phil. Trans. R. Soc. B, 369: 20130540.

Human umbilical cord fibroblasts may be expanded in Dulbecco's Modified Eagle's Medium (e.g. DMEM, SH30081.01, Hyclone) supplemented with human serum (e.g. 20%) and glutamine. Preferably the human cord cells are irradiated. This may be effected using methods known in the art (e.g. Gamma cell, 220 Exel, MDS Nordion 3,500-7500 rads). Once sufficient cells are obtained, they may be frozen (e.g. cryopreserved). For expansion of ESCs, the human cord fibroblasts are typically seeded on a solid surface (e.g. T75 or T 175 flasks) optionally coated with an adherent substrate such as gelatin (e.g. recombinant human gelatin (RhG 100-001, Fibrogen) or human Vitronectin or Laminin 521 (Bio lamina) at a concentration of about 25,000-100,000 cells/cm2 in DMEM (e.g. SH30081.01, Hyclone) supplemented with about 20% human serum (and glutamine). hESCs are typically plated on top of the feeder cells 1-4 days later in a supportive medium (e.g. NUTRISTEM® or NUT(+) with human serum albumin). Additional factors may be added to the medium to prevent differentiation of the ESCs such as bFGF and TGFβ. Once a sufficient amount of hESCs are obtained, the cells may be mechanically disrupted (e.g. by using a sterile tip or a disposable sterile stem cell tool; 14602 Swemed). Alternatively, the cells may be removed by enzymatic treatment (e.g. collagenase A, or TrypLE Select). This process may be repeated several times to reach the necessary amount of hESC. According to some embodiments, following the first round of expansion, the hESCs are removed using TrypLE Select and following the second round of expansion, the hESCs are removed using collagenase A.

The ESCs may be expanded on feeders prior to the differentiation step. Exemplary feeder layer based cultures are described herein above. The expansion is typically effected for at least two days, three days, four days, five days, six days, seven days, eight days, nine days, or ten days. The expansion is effected for at least 1 passage, at least 2 passages, at least 3 passages, at least 4 passages, at least 5 passages, at least 6 passages, at least 7 passages, at least 8 passages, at least 9 passages or at least 10 passages. In some embodiments, the expansion is effected for at least 2 passages to at least 20 passages. In other embodiments, the expansion is effected for at least 2 to at least 40 passages. Following expansion, the pluripotent stem cells (e.g. ESCs) are subjected to directed differentiation using a differentiating agent.

Feeder cell free systems have also been used in ES cell culturing, such systems utilize matrices supplemented with serum replacement, cytokines and growth factors (including IL6 and soluble IL6 receptor chimera) as a replacement for the feeder cell layer. Stem cells can be grown on a solid surface such as an extracellular matrix (e.g., MATRIGELR™, laminin or vitronectin) in the presence of a culture medium—for example the Lonza L7 system, mTeSR, StemPro, XFKSR, E8, NUTRISTEM®). Unlike feeder-based cultures which require the simultaneous growth of feeder cells and stem cells and which may result in mixed cell populations, stem cells grown on feeder-free systems are easily separated from the surface. The culture medium used for growing the stem cells contains factors that effectively inhibit differentiation and promote their growth such as MEF-conditioned medium and bFGF.

In some embodiments, following expansion, the pluripotent ESCs are subjected to directed differentiation on an adherent surface (without intermediate generation of spheroid or embyroid bodies). See, for example, international patent application publication No. WO 2017/072763, incorporated by reference herein in its entirety.

Thus, according to an aspect of the present disclosure, at least 50%, 55%, 60%, 65%, 70%, 75%, 80%, 85%, 90%, 91%, 92%, 93%, 94%, 95%, 96%, 97%, 98%, 99% or 100% of the cells which are subjected to directed differentiation on the adherent surface are undifferentiated ESCs and express markers of pluripotency. For example, at least 50%, 55%, 60%, 65%, 70%, 75%, 80%, 85%, 90%, 91%, 92%, 93%, 94%, 95%, 96%, 97%, 98%, 99% or 100% of the cells are Oct4⁺TRA-1-60⁺. The non-differentiated ESCs may express other markers of pluripotency, such as NANOG, Rex-1, alkaline phosphatase, Sox2, TDGF-beta, SSEA-3, SSEA-4 and/or TRA-1-81.

In one exemplary differentiation protocol, the non-differentiated embryonic stem cells are differentiated towards the RPE cell lineage on an adherent surface using a first differentiating agent and then further differentiated towards RPE cells using a member of the transforming growth factor-B (TGFB) superfamily, (e.g. TGF 1, TGF2, and TGF 3 subtypes, as well as homologous ligands including activin (e.g., activin A, activin B, and activin AB), nodal, anti-mullerian hormone (AMH), some bone morphogenetic proteins (BMP), e.g. BMP2, BMP3, BMP4, BMP5, BMP6, and BMP7, and growth and differentiation factors (GDF)). According to a specific embodiment, the member of the transforming growth factor-B (TGFB) superfamily is activin A—e.g. between 20-200 ng/ml e.g. 100-180 ng/ml.

According to some embodiments, the first differentiating agent is nicotinamide (NA) used at concentrations of between about 1-100 mM, 5-50 mM, 5-20 mM, and for example, 10 mM. According to other embodiments, the first differentiating agent is 3-aminobenzmine.

NA, also known as “niacinamide”, is the amide derivative form of Vitamin B3 (niacin) which is thought to preserve and improve beta cell function. NA has the chemical formula C6H6N20. NA is essential for growth and the conversion of foods to energy, and it has been used in arthritis treatment and diabetes treatment and prevention.

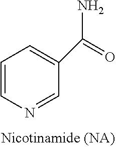

According to some embodiments, the nicotinamide is a nicotinamide derivative or a nicotinamide mimic. The term “derivative of nicotinamide (NA)” as used herein denotes a compound which is a chemically modified derivative of the natural NA. In one embodiment, the chemical modification may be a substitution of the pyridine ring of the basic NA structure (via the carbon or nitrogen member of the ring), via the nitrogen or the oxygen atoms of the amide moiety. When substituted, one or more hydrogen atoms may be replaced by a substituent and/or a substituent may be attached to a N atom to form a tetravalent positively charged nitrogen. Thus, the nicotinamide of the present invention includes a substituted or non-substituted nicotinamide. In another embodiment, the chemical modification may be a deletion or replacement of a single group, e.g. to form a thiobenzamide analog of NA, all of which being as appreciated by those versed in organic chemistry. The derivative in the context of the invention also includes the nucleoside derivative of NA (e.g. nicotinamide adenine). A variety of derivatives of NA are described, some also in connection with an inhibitory activity of the PDE4 enzyme (WO 03/068233; WO 02/060875; GB2327675A), or as VEGF-receptor tyrosine kinase inhibitors (WOO 1/55114). For example, the process of preparing 4-aryl-nicotinamide derivatives (WO 05/014549). Other exemplary nicotinamide derivatives are disclosed in WOO 1/55114 and EP2128244.

Nicotinamide mimics include modified forms of nicotinamide, and chemical analogs of nicotinamide which recapitulate the effects of nicotinamide in the differentiation and maturation of RPE cells from pluripotent cells. Exemplary nicotinamide mimics include benzoic acid, 3-aminobenzoic acid, and 6-aminonicotinamide. Another class of compounds that may act as nicotinamide mimics are inhibitors of poly(ADP-ribose) polymerase (PARP). Exemplary PARP inhibitors include 3-aminobenzamide, Iniparib (BSI 201), Olaparib (AZD-2281), Rucaparib (AG014699, PF-01367338), Veliparib (ABT-888), CEP 9722, MK 4827, and BMN-673.

Additional contemplated differentiation agents include for example noggin, antagonists of Wnt (Dkkl or IWR1e), nodal antagonists (Lefty-A), retinoic acid, taurine, GSK3b inhibitor (CHIR99021) and notch inhibitor (DAPT).

According to certain embodiments, the differentiation is effected as follows: (a) culture of ESCs in a medium comprising a first differentiating agent (e.g. nicotinamide); and (b) culture of cells obtained from step a) in a medium comprising a member of the TGFB superfamily (e.g. activin A) and the first differentiating agent (e.g. nicotinamide).

Step (a) may be effected in the absence of the member of the TGFβ superfamily (e.g. activin A).

In some embodiments, the medium in step (a) is completely devoid of a member of the TGFβ superfamily. In other embodiments, the level of TGFI3 superfamily member in the medium is less than 20 ng/ml, 10 ng/ml, 1 ng/ml or even less than 0.1 ng/ml.

The above described protocol may be continued by culturing the cells obtained in step (b) in a medium comprising the first differentiating agent (e.g. nicotinamide), but devoid of a member of the TGFI3 superfamily (e.g. activin A). This step is referred to herein as step (b*).

The above described protocol is now described in further detail, with additional embodiments. Step (a): The differentiation process is started once sufficient quantities of ESCs are obtained. The cells may be removed from the cell culture (e.g. by using collagenase A, dispase, TrypLE select, EDTA) and plated onto a non-adherent substrate (e.g. cell culture plate such as Hydrocell or an agarose-coated culture dish, or petri bacteriological dishes) in the presence of nicotinamide (and the absence of activin A). Exemplary concentrations of nicotinamide are between 0.01-100 mM, 0.1-100 mM, 0.1-50 mM, 5-50 mM, 5-20 mM, and 10 mM. Once the cells are plated onto the non-adherent substrate (e.g. cell culture plate), the cell culture may be referred to as a cell suspension, preferably free-floating clusters in a suspension culture, i.e. aggregates of cells derived from human embryonic stem cells (hESCs). The cell clusters do not adhere to any substrate (e.g. culture plate, carrier). Sources of free floating stem cells were previously described in WO 06/070370, which is herein incorporated by reference in its entirety. This stage may be effected for a minimum of 1 day, more preferably two days, three days, 1 week or even 14 days. Preferably, the cells are not cultured for more than 3 weeks in suspension together with the nicotinamide e.g. between 0.01-100 mM, 0.1-100 mM, 0.1-50 mM, 5-50 mM, 5-20 mM, e.g. 10 mM (and in the absence of activin A). In one embodiment, the cells are cultured for 6-8 days in suspension together with the nicotinamide e.g. between 0.01-100 mM, 0.1-100 mM, 0.1-50 mM, 5-50 mM, 5-20 mM, e.g. 10 mM (and in the absence of activin A).

According to some embodiments, when the cells are cultured on the non-adherent substrate, e.g. cell culture plates, the atmospheric oxygen conditions are 20%. However, manipulation of the atmospheric oxygen conditions is also contemplated such that the atmospheric oxygen percent is less than about 20%, 15%, 10%, 9%, 8%, 7%, 6% or even less than about 5% (e.g. between 1%-20%, 1%-10% or 0-5%). According to other embodiments, the cells are cultured on the non-adherent substrate initially under normal atmospheric oxygen conditions and then lowered to less than normal atmospheric oxygen conditions.

Examples of non-adherent cell culture plates include those manufactured by Nunc (e.g. Hydrocell Cat No. 174912), etc.

Typically, the clusters comprise at least 50-500,000, 50-100,000, 50-50,000, 50-10,000, 50-5000, 50-1000 cells. According to one embodiment, the cells in the clusters are not organized into layers and form irregular shapes. In one embodiment, the clusters are substantially devoid of pluripotent embryonic stem cells. In another embodiment, the clusters comprise small amounts of pluripotent embryonic stem cells (e.g. no more than 5%, or no more than 3% (e.g. 0.01-2.7%) cells that co-express OCT4 and TRA-1-60 at the protein level). Typically, the clusters comprise cells that have been partially differentiated under the influence of nicotinamide. Such cells primarily express neural and retinal precursor markers such as PAX6, Rax, Six3 and/or CHX10.

The clusters may be dissociated using enzymatic or non-enzymatic methods (e.g., mechanical) known in the art. According to some embodiments, the cells are dissociated such that they are no longer in clusters—e.g. aggregates or clumps of 2-100,000 cells, 2-50,000 cells, 2-10,000 cells, 2-5000 cells, 2-1000 cells, 2-500 cells, 2-100 cells, 2-50 cells. According to a particular embodiment, the cells are in a single cell suspension.

The cells (e.g. dissociated cells) can then be plated on an adherent substrate and cultured in the presence of nicotinamide e.g. between 0.01-100 mM, 0.1-100 mM, 0.1-50 mM, 5-50 mM, 5-20 mM, and for example, 10 mM (and in the absence of activin A). This stage may be effected for a minimum of 1 day, more preferably two days, three days, 1 week or even 14 days. Preferably, the cells are not cultured for more than 3 weeks in the presence of nicotinamide (and in the absence of activin). In an exemplary embodiment, this stage is effected for 6-7 days.

According to other embodiments, when the cells are cultured on the adherent substrate e.g. laminin, the atmospheric oxygen conditions are 20%. They may be manipulated such that the atmospheric oxygen percentage is less than about 20%, 15%, 10%, more preferably less than about 9%, less than about 8%, less than about 7%, less than about 6% and more preferably about 5% (e.g. between 1%-20%, 1%-10% or 0-5%).

According to some embodiments, the cells are cultured on the adherent substrate initially under normal atmospheric oxygen conditions and subsequently the oxygen is lowered to less than normal atmospheric oxygen conditions.

Examples of adherent substrates or a mixture of substances could include but are not limited to fibronectin, laminin, polyD-lysine, collagen and gelatin.

Step (b): Following the first stage of directed differentiation, (step a; i.e. culture in the presence of nicotinamide (e.g. between 0.01-100 mM, 0.1-100 mM, 0.1-50 mM, 5-50 mM, 5-20 mM, e.g. 10 mM), the partially-differentiated cells may then be subjected to a further stage of differentiation on an adherent substrate by culturing in the presence of activin A (e.g. 0.01-1000 ng/ml, 0.1-200 ng/ml, 1-200 ng/ml—for example 140 ng/ml, 150 ng/ml, 160 ng/ml or 180 ng/ml). Thus, activin A may be added at a final molarity of 0.1 pM-10 nM, 10 pM-10 nM, 0.1 nM-10 nM, 1 nM-10 nM, for example 5.4 nM.

Nicotinamide may be added at this stage as well (e.g. between 0.01-100 mM, 0.1-100 mM, 0.1-50 mM, 5-50 mM, 5-20 mM, e.g. 10 mM). This stage may be effected for 1 day to 10 weeks, 3 days to 10 weeks, 1 week to 10 weeks, one week to eight weeks, one week to four weeks, for example for at least one day, at least two days, at least three days, at least 5 days, at least one week, at least 9 days, at least 10 days, at least two weeks, at least three weeks, at least four weeks, at least five weeks, at least six weeks, at least seven weeks, at least eight weeks, at least nine weeks, at least ten weeks.

According to some embodiments, this stage is effected for about eight days to about two weeks. This stage of differentiation may be effected at low or normal atmospheric oxygen conditions, as detailed herein above.

Step (b*): Following the second stage of directed differentiation (i.e. culture in the presence of nicotinamide and activin A on an adherent substrate; step (b), the further differentiated cells are optionally subjected to a subsequent stage of differentiation on the adherent substrate culturing in the presence of nicotinamide (e.g. between 0.01-100 mM, 0.1-100 mM, 0.1-50 mM, 5-50 mM, 5-20 mM, e.g. 10 mM), in the absence of activin A. This stage may be effected for at least one day, 2, days, 5 days, at least one week, at least two weeks, at least three weeks or even four weeks. This stage of differentiation may also be carried out at low or normal atmospheric oxygen conditions, as detailed herein above.

The basic medium in which the ESCs are differentiated is any known cell culture medium known in the art for supporting cell growth in vitro, typically, a medium comprising a defined base solution, which includes salts, sugars, amino acids and any other nutrients required for the maintenance of the cells in the culture in a viable state. According to a specific embodiment, the basic medium is not a conditioned medium. Non-limiting examples of commercially available basic media that may be utilized in accordance with the invention comprise NUTRISTEM® (without bFGF and TGF for ESC differentiation, with bFGF and TGF for ESC expansion), NEUROBASAL™, KO-DMEM, DMEM, DMEM/F12, CELLGRO™ Stem Cell Growth Medium, or X-VIVO™. The basic medium may be supplemented with a variety of agents as known in the art dealing with cell cultures. The following is a non-limiting reference to various supplements that may be included in the culture to be used in accordance with the present disclosure: serum or with a serum replacement containing medium, such as, without being limited thereto, knock out serum replacement (KOSR), NUTRIDOMA-CS, TCH™, N2, N2 derivative, or B27 or a combination; an extracellular matrix (ECM) component, such as, without being limited thereto, fibronectin, laminin, collagen and gelatin. The ECM may then be used to carry the one or more members of the TGFβ superfamily of growth factors; an antibacterial agent, such as, without being limited thereto, penicillin and streptomycin; and non-essential amino acids (NEAA), neurotrophins which are known to play a role in promoting the survival of SCs in culture, such as, without being limited thereto, BDNF, NT3, NT4.

According to some embodiments, the medium used for differentiating the ESCs is NUTRISTEM® medium (Biological Industries, 06-5102-01-1A).

According to some embodiments, differentiation and expansion of ESCs is effected under xeno free conditions. According other embodiments, the proliferation/growth medium is substantially devoid of xeno contaminants i.e., free of animal derived components such as serum, animal derived growth factors and albumin. Thus, according to these embodiments, the culturing is performed in the absence of xeno contaminants. Other methods for culturing ESCs under xeno free conditions are provided in U.S. Patent Application No. 20130196369, the contents of which are incorporated herein by reference in its entirety.

The preparations comprising RPE cells may be prepared in accordance with Good Manufacturing Practices (GMP) (e.g., the preparations are GMP-compliant) and/or current Good Tissue Practices (GTP) (e.g., the preparations may be GTP-compliant).

During differentiation steps, the embryonic stem cells may be monitored for their differentiation state. Cell differentiation can be determined upon examination of cell or tissue-specific markers which are known to be indicative of differentiation.

Tissue/cell specific markers can be detected using immunological techniques well known in the art [Thomson J A et al., (1998). Science 282: 1145-7]. Examples include, but are not limited to, flow cytometry for membrane-bound or intracellular markers, immunohistochemistry for extracellular and intracellular markers and enzymatic immunoassay, for secreted molecular markers.

Following the stages of differentiation described herein above, a mixed cell population can be obtained comprising both pigmented and non-pigmented cells. According to this aspect, the cells of the mixed cell population are removed from the plate. In some embodiments, this is effected enzymatically (e.g. using trypsin, (TrypLE Select); see for example, international patent application publication No. WO 2017/021973, incorporated by reference herein in its entirety). According to this aspect of the present invention, at least 10%, 20%, 30%, at least 40%, at least 50%, at least 60%, at least 70% of the cells which are removed from the culture (and subsequently expanded) are non-pigmented cells. In other embodiments, this is effected mechanically—e.g. using a cell scraper. In yet other embodiments, this is effected chemically (e.g., by EDTA). Combinations of enzymatic and chemical treatment are also contemplated. For example, EDTA and enzymatic treatments can be used. Furthermore, at least 10%, 20% or even 30% of the cells which are removed from the culture (and subsequently expanded) may be pigmented cells.

According to an aspect of the present disclosure, at least 50%, 60%, 70%, 80%, 90%, 95%, 100% of all the cells in the culture are removed and subsequently expanded.

Expansion of the mixed population of cells may be effected on an extra cellular matrix, e.g. gelatin, collagen I, collagen IV, laminin (e.g. laminin 521), fibronectin and poly-D-lysine. For expansion, the cells may be cultured in serum-free KOM, serum comprising medium (e.g. DMEM with 20% human serum) or NUTRISTEM® medium (06-5102-01-1A, Biological Industries). Under these culture conditions, after passaging under suitable conditions, the ratio of pigmented cells to non-pigmented cells increases such that a population of purified RPE cells is obtained. Such cells show the characteristic polygonal shape morphology and pigmentation of RPE cells.

In one embodiment, the expanding is effected in the presence of nicotinamide (e.g. between 0.01-100 mM, 0.1-100 mM, 0.1-50 mM, 5-50 mM, 5-20 mM, e.g. 10 mM), and in the absence of activin A.

The mixed population of cells may be expanded in suspension (with or without a micro-carrier) or in a monolayer. The expansion of the mixed population of cells in monolayer cultures or in suspension culture may be modified to large scale expansion in bioreactors or multi/hyper stacks by methods well known to those versed in the art.

According to some embodiments, the expansion phase is effected for at least one to 20 weeks, at least 2 weeks, at least 3 weeks, at least 4 weeks, at least 5 weeks, at least 6 weeks, at least 7 weeks, at least 8 weeks, at least 9 weeks or even 10 weeks. Preferably, the expansion phase is effected for 1 week to 10 weeks, more preferably 2 weeks to 10 weeks, more preferably, 3 weeks to 10 weeks, more preferably 4 weeks to 10 weeks, or 4 weeks to 8 weeks.

According to still other embodiments, the mixed population of cells are passaged at least 1 time during the expansion phase, at least twice during the expansion phase, at least three times during the expansion phase, at least four times during the expansion phase, at least five times during the expansion phase, or at least six times during the expansion phase.

The present inventors have shown that when cells are collected enzymatically, it is possible to continue the expansion for more than 8 passages, more than 9 passages and even more than 10 passages (e.g. 11-15 passages). The number of total cell doublings can be increased to greater than 30, e.g. 31, 32, 33, 34 or more. (See international patent application publication number WO 2017/021973, incorporated herein by reference in its entirety).

The population of RPE cells generated according to the methods described herein may be characterized according to a number of different parameters. Thus, for example, the RPE cells obtained may be polygonal in shape and pigmented.

It will be appreciated that the cell populations and cell compositions disclosed herein are generally devoid of undifferentiated human embryonic stem cells. According to some embodiments, less than 1:250,000 cells are Oct4+TRA-1-60+ cells, as measured for example by FACS. The cells may also have down regulated (by more than 5,000 fold) expression of GDF3 or TDGF as measured by PCR. The RPE cells of this aspect, do not substantially express embryonic stem cell markers. Said one or more embryonic stem cell markers may comprise OCT-4, NANOG, Rex-1, alkaline phosphatase, Sox2, TDGF-beta, SSEA-3, SSEA-4, TRA-1-60, and/or TRA-1-81.

The therapeutic RPE cell preparations may be substantially purified, with respect to non-RPE cells, comprising at least about 75%, 80%, 85%, 90%, 91%, 92%, 93%, 94%, 95%, 96%, 97%, 98%, 99%, or 100% RPE cells. The RPE cell preparations may be essentially free of non-RPE cells or consist of RPE cells. For example, the substantially purified preparation of RPE cells may comprise less than about 25%, 20%, 15%, 10%, 9%, 8%, 7%, 6%, 5%, 4%, 3%, 2%, or 1% non-RPE cell type. For example, the RPE cell preparation may comprise less than about 25%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% non-RPE cells.

The RPE cell preparations may be substantially pure, both with respect to non-RPE cells and with respect to RPE cells of other levels of maturity. The preparations may be substantially purified, with respect to non-RPE cells, and enriched for mature RPE cells. For example, in RPE cell preparations enriched for mature RPE cells, at least about 30%, 40%, 45%, 50%, 55%, 60%, 65%, 70%, 75%, 80%, 85%, 90%, 91%, 92%, 93%, 94%, 95%, 96%, 97%, 98%, 99%, 99%, or 100% of the RPE cells are mature RPE cells. The preparations may be substantially purified, with respect to non-RPE cells, and enriched for differentiated RPE cells rather than mature RPE cells. For example, at least about 30%, 40%, 45%, 50%, 55%, 60%, 65%, 70%, 75%, 80%, 85%, 90%, 91%, 92%, 93%, 94%, 95%, 96%, 97%, 98%, 99%, or 100% of the RPE cells may be differentiated RPE cells rather than mature RPE cells.

The preparations described herein may be substantially free of bacterial, viral, or fungal contamination or infection, including but not limited to the presence of HIV I, HIV 2, HBV, HCV, HAV, CMV, HTLV 1, HTLV 2, parvovirus B19, Epstein-Barr virus, or herpesvirus 1 and 2, SV40, HHVS, 6, 7, 8, CMV, polyoma virus, HPV, Enterovirus. The preparations described herein may be substantially free of mycoplasma contamination or infection.

Another way of characterizing the cell populations disclosed herein is by marker expression. Thus, for example, at least 80%, 85%, 90%, 95% or 100% of the cells may express Bestrophin 1, as measured by immunostaining. According to one embodiment, between 80-100% of the cells express bestrophin 1.

According to other embodiments, at least 80%, 85%, 87%, 89%, 90%, 95%, 97% or 100% of the cells express Microphthalmia-associated transcription factor (MITF), as measured by immunostaining. For example, between 80-100% of the cells express MITF.

According to other embodiments, at least 80%, 85%, 87%, 89%, 90%, 95%, 97% or 100% of the cells express both Microphthalmia-associated transcription factor (MITF) and bestrophin 1, as measured by immunostaining. For example, between 80-100% of the cells co-express MITF and bestrophin 1.

According to other embodiments, at least 80%, 85%, 87%, 89%, 90%, 95%, 97% or 100% of the cells express both Microphthalmia-associated transcription factor (MITF) and ZO-1, as measured by immunostaining. For example, between 80-100% of the cells co-express MITF and ZO-1.

According to other embodiments, at least 80%, 85%, 87%, 89%, 90%, 95%, 97% or 100% of the cells express both ZO-1 and bestrophin 1, as measured by immunostaining. For example, between 80-100% of the cells co-express ZO-1 and bestrophin 1.

According to another embodiment, at least 50%, 60% 70% 80%, 85%, 87%, 89%, 90%, 95%, 97% or 100% of the cells express paired box gene 6 (PAX-6) as measured by immunostaining or FACS. For example, at least between 50% and 100% of the cells express paired box gene 6 (PAX-6).

According to another embodiment, at least 80%, 85%, 87%, 89%, 90%, 95%, 97% or 100% of the cells express cellular retinaldehyde binding protein (CRALBP), as measured by immunostaining. For example, between 80-100% of the cells express CRALBP.

According to another embodiment, at least 80%, 85%, 87%, 89%, 90%, 95%, 97% or 100% of the cells express cellular Melanocytes Lineage-Specific Antigen GP100 (PMEL17), as measured by immunostaining. For example, between about 80-100% of the cells express PMEL17.

The RPE cells may co-express markers indicative of terminal differentiation, e.g. bestrophin 1, CRALBP and/or RPE65. According to one embodiment, at least 95%, at least 96%, at least 97%, at least 98%, at least 99%, at least 100% or even between about 50% to 100% of the cells of the RPE cell populations obtained co-express both premelanosome protein (PMEL17) and cellular retinaldehyde binding protein (CRALBP).

According to a particular embodiment, the cells coexpress PMEL17 (SwissProt No. P40967) and at least one polypeptide selected from the group consisting of cellular retinaldehyde binding protein (CRALBP; SwissProt No. P12271), lecithin retinol acyltransferase (LRAT; SwissProt No. 095327) and sex determining region Y-box 9 (SOX 9; P48436).

According to a particular embodiment, at least 80% of the cells of the population express detectable levels of PMEL17 and one of the above mentioned polypeptides (e.g. CRALBP), more preferably at least 85% of the cells of the population express detectable levels of PMEL17 and one of the above mentioned polypeptides (e.g. CRALBP), more preferably at least 90% of the cells of the population express detectable levels of PMEL17 and one of the above mentioned polypeptides (e.g. CRALBP), more preferably at least 95% of the cells of the population express detectable levels of PMEL17 and one of the above mentioned polypeptides (e.g. CRALBP), more preferably 100% of the cells of the population express detectable levels of PMEL17 and one of the above mentioned polypeptides (e.g. CRALBP as assayed by a method known to those of skill in the art (e.g. FACS).

According to another embodiment, the level of CRALBP and one of the above-mentioned polypeptides (e.g. PMEL17) coexpression (e.g. as measured by the mean fluorescent intensity) is increased by at least two fold, more preferably at least 3 fold, more preferably at least 4 fold and even more preferably by at least 5 fold, at least 10 fold, at least 20 fold, at least 30 fold, at least 40 fold, at least 50 fold as compared to non-differentiated ESCs.

In one embodiment, the RPE are terminally differentiated and do not generally express Pax6. In another embodiment, the RPE cells are terminally differentiated and generally express Pax6.

The RPE cells described herein may also act as functional RPE cells after transplantation wherein the RPE cells may form a monolayer between the neurosensory retina and the choroid in the patient receiving the transplanted cells. The RPE cells may also supply nutrients to adjacent photoreceptors and dispose of shed photoreceptor outer segments by phagocytosis.

According to one embodiment, the trans-epithelial electrical resistance of the cells in a monolayer is greater than 100 ohms.

Preferably, the trans-epithelial electrical resistance of the cells is greater than 150, 200, 250, 300, 300, 400, 500, 600, 700, 800 or even greater than 900 ohms.

Devices for measuring trans-epithelial electrical resistance (TEER) are known in the art and include for example EVOM2 Epithelial Voltohmmeter, (World Precision Instruments).

Following the expansion phase, cell populations comprising RPE cells are obtained whereby at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% thereof are CRALBP+ PMEL1 7+.

It would be well appreciated by those versed in the art that the derivation of RPE cells is of great benefit. They may be used as an in vitro model for the development of new drugs to promote their survival, regeneration and function. RPE cells may serve for high throughput screening for compounds that have a toxic or regenerative effect on RPE cells. They may be used to uncover mechanisms, new genes, soluble or membrane-bound factors that are important for the development, differentiation, maintenance, survival and function of photoreceptor cells.

The RPE described herein cells may also serve as an unlimited source of RPE cells for transplantation, replenishment and support of malfunctioning or degenerated RPE cells in retinal degenerations and other degenerative disorders. Furthermore, genetically modified RPE cells may serve as a vector to carry and express genes in the eye and retina after transplantation.

In certain embodiments, RPE cell compositions may be produced according to following methods: (1) culturing hESCs on hUCFs in CW plates for 2 weeks in NUT+ with human serum albumin (HSA), (2) mechanical passaging to expand the hESCs on hUCFs in CW plates for between four to five weeks (or until desired amount of cells) in NUT+ with HSA, (3) continue to expand hESC colonies (using for example, collagenase) on hUCFs in 6 cm plates for an additional week in NUT+ with HSA, (4) prepare spheroid bodies (SB) by transferring colonies from about five 6 cm plates into 1 HydroCell for about one week in NUT− with nicotinamide (NIC), (5) flattening of SBs on Lam511 may be carried out by transferring the SBs to 2-3 wells of a 6-well plate for about one week in NUT− with NIC, (6) culture adherent cells on Lam511 in NUT− with NIC and Activin for about one to two weeks and replace media with NUT− with NIC and culture for between one and three weeks, (7) enrich for pigmented cells using enzymes, such as TrypLE Select for example, (8) expand RPE cells on gelatin in flasks for between about two to nine weeks (replacing media) in 20% human serum and NUT-, and (9) harvest RPE cells.

Harvesting of the expanded population of RPE cells may be effected using methods known in the art (e.g. using an enzyme such as trypsin, or chemically using EDTA, etc). In some embodiments, the RPE cells may be washed using an appropriate solution, such as PBS or BSS plus. In other embodiments, the RPE cells may be filtered prior to formulation of the RPE cell compositions for cryopreservation and administration to a subject directly after thawing.

Following harvesting, the expanded population of RPE cells can be formulated at a specific therapeutic dose (e.g., number of cells) and cryopreserved for shipping to the clinic. The ready to administer (RTA) RPE cell therapy composition can then be administered directly after thawing without further processing. Examples of media suitable for cryopreservation include but are not limited to 90% Human Serum/10% DMSO, Media 3 10% (CS10), Media 2 5% (CS5) and Media 1 2% (CS2), Stem Cell Banker, PRIME XV® FREEZIS, HYPOTHERMASOL®, Trehalose, etc.

RPE cells formulated in cryopreservation media appropriate for post thaw ready to administer (RTA) applications may comprise RPE cells suspended in adenosine, dextran □40, lactobionic acid, HEPES (N□(2□Hydroxyethyl) piperazine □N′□(2□ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), and water. An example of this cryopreservation media is available commercially under the tradename, CRYOSTOR® and is manufactured by BioLife Solutions, Inc.

In further embodiments, the cryopreservation media includes: a purine nucleoside (e.g., adenosine), a branched glucan (e.g., dextran □40), a zwitterionic organic chemical buffering agent (e.g., HEPES (N□(2□Hydroxyethyl) piperazine □N′□(2□ethanesulfonic acid))), and a cell tolerable polar aprotic solvent (e.g., dimethyl sulfoxide (DMSO). In still further embodiments, one or more of the purine nucleoside, branched glucan, buffering agent, and the polar aprotic solvent are generally recognized as safe by the US FDA.

In some embodiments, the cryopreservation media further includes one or more of: a sugar acid (e.g., lactobionic acid), one or more of a base (e.g., sodium hydroxide, potassium hydroxide), an antioxidant (e.g., L-glutathione), one or more halide salt (e.g., potassium chloride, sodium chloride, magnesium chloride), a basic salt (e.g., potassium bicarbonate), phosphate salt (e.g., potassium phosphate, sodium phosphate, potassium phosphate), one or more sugars (e.g., dextrose, sucrose), sugar alcohol, (e.g., mannitol), and water.

In other embodiments, one or more of the sugar acid, base, halide salt, basic salt, antioxidant, phosphate salt, sugars, sugar alcohols are generally recognized as safe by the US FDA.

DMSO can be used as a cryoprotective agent to prevent the formation of ice crystals, which can kill cells during the cryopreservation process. In some embodiments, the cryopreservable RPE cell therapy composition comprises between about 0.1% and about 2% DMSO (v/v). In some embodiments, the RTA RPE cell therapy composition comprises between about 1% and about 20% DMSO. In some embodiments, the RTA RPE cell therapy composition comprises about 2% DMSO. In some embodiments, the RTA RPE cell therapy composition comprises about 5% DMSO.

In some embodiments, RPE cell therapies formulated in cryopreservation media appropriate for post thaw ready to administer applications may comprise RPE cells suspended in cryopreservation media that does not contain DMSO. For example, RTA RPE cell therapy compositions may comprise RPE cells suspended in Trolox, Na+, K+, Ca2+, Mg2+, cl-, H2P04-, HEPES, lactobionate, sucrose, mannitol, glucose, dextran-40, adenosine, glutathione without DMSO (dimethyl sulfoxide, (CH3)₂SO) or any other dipolar aprotic solvents. An example of this cryopreservation media is available commercially under the tradename, HYPOTHERIVIOSOL® or HYPOTHERMOSOL®-FRS and is also manufactured by BioLife Solutions, Inc. In other embodiments, RPE cell compositions formulated in cryopreservation media appropriate for post thaw ready to administer applications may comprise RPE cells suspended in Trehalose.

The RTA RPE cell therapy composition may optionally comprise additional factors that support RPE engraftment, integration, survival, potency, etc. In some embodiments, the RTA RPE cell therapy composition comprises activators of function of the RPE cell preparations described herein. In some embodiments, the RTA RPE cell therapy composition comprises nicotinamide. In some embodiments, the RTA RPE cell therapy composition comprises nicotinamide at a concentration of between about 0.01-100 mM, 0.1-100 mM, 0.1-50 mM, 5-50 mM, 5-20 mM, e.g. 10 mM. In other embodiments, the RTA RPE cell therapy composition comprises retinoic acid. In some embodiments, the RTA RPE cell therapy composition comprises retinoic acid at a concentration of between about 0.01-100 mM, 0.1-100 mM, 0.1-50 mM, 5-50 mM, 5-20 mM, e.g. 10 mM.

In some embodiments, the RTA RPE cell therapy composition may be formulated to include activators of various integrins that have been shown to increase the adherence of the RPE cell preparations, such as those described herein, to the Brunch's membrane. For example, in some embodiments, the RTA RPE cell therapy composition comprises extracellular manganese (Mn2+) at a concentration of between about 5 μM and 1,000 μM. In other embodiments, the RTA RPE cell therapy composition comprises the conformation-specific monoclonal antibody, TS2/16.

In other embodiments, the RTA RPE cell therapy composition may also be formulated to include activators of RPE cell immune regulatory activity.

In some embodiments, the RTA RPE cell therapy composition may include a ROCK inhibitor.

In some embodiments, RPE cell therapies formulated in cryopreservation media appropriate for post thaw ready to administer applications may comprise one or more immunosuppressive compounds. In certain embodiments, RPE cell therapies formulated in cryopreservation media appropriate for post thaw ready to administer applications may comprise one or more immunosuppressive compounds that are formulated for slow release of the one or more immunosuppressive compounds. Immunosuppressive compounds for use with the formulations described herein may belong to the following classes of immunosuppressive drugs: Glucocorticoids, Cytostatics (e.g. alkylating agent or antimetabolite), antibodies (polyclonal or monoclonal), drugs acting on immunophilins (e.g. cyclosporin, Tacrolimus or Sirolimus). Additional drugs include interferons, opioids, TNF binding proteins, mycophenolate and small biological agents. Examples of immunosuppressive drugs include: mesenchymal stem cells, anti-lymphocyte globulin (ALG) polyclonal antibody, anti-thymocyte globulin (ATG) polyclonal antibody, azathioprine, BAS 1L1×1MAB® (anti-I L-2Ra receptor antibody), cyclosporin (cyclosporin A), DACLIZUMAB® (anti-I L-2Ra receptor antibody), everolimus, mycophenolic acid, RITUX 1MAB® (anti-CD20 antibody), sirolimus, tacrolimus, Tacrolimus and or Mycophenolate mofetil.

The RPE cells may be transplanted in various forms. For example, the RPE cells may be introduced into the target site in the form of single cell suspension, with matrix or adhered onto a matrix or a membrane, extracellular matrix or substrate such as a biodegradable polymer or a combination. The RPE cells may also be printed onto a matrix or scaffold. The RPE cells may also be transplanted together (co-transplantation) with other retinal cells, such as with photoreceptors. The effectiveness of treatment may be assessed by different measures of visual and ocular function and structure, including, among others, best corrected visual acuity (BCVA), retinal sensitivity to light as measured by perimetry or microperimetry in the dark and light-adapted states, full-field, multi-focal, focal or pattern electroretinography 5 ERG), contrast sensitivity, reading speed, color vision, clinical biomicroscopic examination, fundus photography, optical coherence tomography (OCT), fundus auto-fluorescence (FAF), infrared and multicolor imaging, fluorescein or ICG angiography, adoptive optics and additional means used to evaluate visual function and ocular structure.

In certain embodiments, treating or slowing the progression, maintain stasis of or reversing retinal disease is demonstrated by microperimetry assessed recovery of vision, wherein microperimetry assessed recovery of vision comprises a correlation between retinal sensitivity on microperimetry and EZ defect as compared to a baseline, an age-matched, sex-matched control, or a fellow eye of the subject. In certain embodiments, treating or slowing the progression, maintain stasis of or reversing retinal disease is demonstrated by microperimetry assessed recovery of vision, wherein there is a correlation of ellipsoid zone (EZ) defects on spectral-domain optical coherence tomography (SD-OCT) with retinal sensitivity loss on macular integrity assessment (MAIA) microperimetry. See Invest Ophthalmol Vis Sci. 2017 May 1; 58(6):B10291-BI0299. doi: 10.1167/iovs.17-21834, “Correlation Between Macular Integrity Assessment and Optical Coherence Tomography Imaging of Ellipsoid Zone in Macular Telangiectasia Type 2”; Mukherjee D. et al., which is herein incorporated by reference in its entirety.

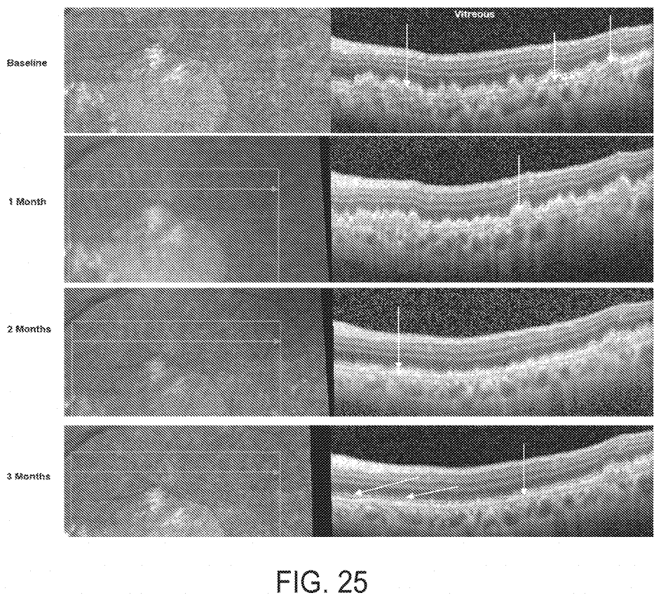

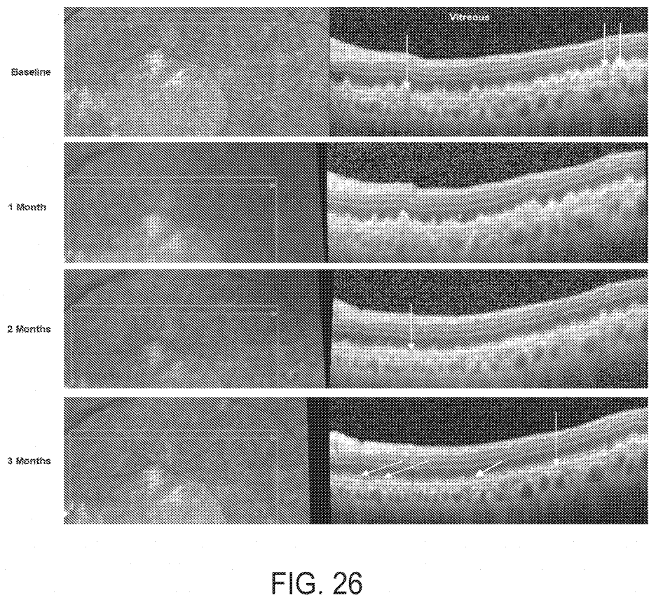

In other embodiments, topographic maps, for example, orthogonal topographic (en face) maps, of the ellipsoid zone were generated from OCT volume scans, for example, Heidelberg Spectralis OCT volume scans (15×10° area, 30-μm B-scan intervals) or Zeiss Cirrus HD-OCT 4000 512×128 cube scans, to demonstrate treating or slowing the progression, maintain stasis of or reversing retinal disease, by comparing the maps to age-matched, sex-matched control, a baseline of the subject or a fellow eye of the subject. There is a correlation between organization of the EZ and retinal sensitivity. After administration of the RPE cells, the EZ zone organizes and retinal sensitivity improves. For example, see FIGS. 25 and 26, at 3 months. See for example, Retina, 2018 January; 38 Suppl 1:S27-S32. “Correlation Of Structural And Functional Outcome Measures In A Phase One Trial Of Ciliary Neurotrophic Factor In Type 2 Idiopathic Macular Telangiectasia,” Sallo F B, et al., which is incorporated by reference in its entirety.

In certain embodiments, treating or slowing the progression, maintain stasis of or reversing retinal disease is demonstrated by OCT-A, as compared to compared to age-matched, sex-matched controls, a baseline of the subject or a fellow eye before and after administration.

For example, using spectral-domain (SD)-OCT and OCT-A imaging and analyzing SD-OCT data using, for example, OCT EZ-mapping to obtain linear, area, and volumetric measurements of the EZ-retinal pigment epithelium (RPE) complex across the macular cube. OCT-A retinal capillary density can be measured using, for example, the Optovue Avanti split-spectrum amplitude-decorrelation angiography algorithm. EZ-RPE parameters are compared to age-matched, sex-matched controls, a baseline of the subject or a fellow eye.

In one embodiment, after administration, the EZ-RPE central foveal mean thickness improves, the EZ-RPE central foveal thickness improves, and EZ-RPE central subfield volume improves. EZ-RPE thickness, area, and volume are correlated with improved visual acuity to measure treatment response. Each of these measurements is inversely correlated with visual acuity. See FIGS. 25 and 26, wherein from baseline to 3 months, there is a decrease in the volume of the EZ. See, for example, methods outlined in, Invest Ophthalmol Vis Sci. 2017 Jul. 1; 58(9):3683-3689, “OCT Angiography and Ellipsoid Zone Mapping of Macular Telangiectasia Type 2 From the AVATAR Study,” Runkle A P., et al, which is incorporated by reference in its entirety.

In one embodiment, recovery, for example, is the subjective assessment that one or more of the following are becoming more organized, including the, external limiting membrane, myoid zone (inner segments of photoreceptors), ellipsoid zone (IS/OS Junction), outer segments of the photoreceptors, loss of drusen, and disappearance of reticular pseudo-drusen. Recovery may also comprise the subjective assessment that one or more of the basic foundational layers of the retina are becoming more organized. As used herein, the basic foundational layers of the retina becoming more organized comprise one or more of the external limiting membrane, myoid zone (inner segments of photoreceptors), ellipsoid zone (IS/OS Junction), and outer segments of the photoreceptors. As seen in FIGS. 25 and 26, the organization is demonstrated, for example, by the decrease in volume of the structures of the EZ, see for example the comparison of the baseline and months 2 and 3. For example, the volume of the EZ is decreased by at least 2%, by at least 5%, by at least 10%.

In one embodiment, the ellipsoid zone analysis demonstrates organization of the EZ by a decrease in the EZ volume as compared to an age-matched, sex-matched control, a baseline or a fellow eye. hi another embodiment, the decrease in the EZ volume comprises at least 2% or at least 5% or at least 7% or at least 10%, or between 1 and 5% or between 1 and 10% or between 1 and 50% or between 10 and 50%. In another embodiment, the organization of the EZ is demonstrated, for example, by the decrease in volume of the structures of the EZ, see for example the comparison of the baseline and months 2 and 3. For example, the volume of the EZ is decreased by at least 2%, by at least 5%, by at least 10%.

In one embodiment, recovery comprises one or more of EZ-RPE central foveal mean thickness improvement, the EZ-RPE central foveal thickness improvement, and EZ-RPE central subfield volume improvement. EZ-RPE thickness, area, and volume are correlated with improved visual acuity to measure treatment response. Each of these measurements is inversely correlated with visual acuity.

RTA RPE cell therapies formulated according to the present disclosure do not require the use of GMP facilities for preparation of the final dose formulation prior to injection into a subject's eye. The RTA RPE cell therapy formulations described herein may be cryopreserved in a non-toxic cryosolution that comprises the final dose formulation which can be shipped directly to the clinical site. When needed, the formulation can be thawed and administered into the subject's eye without having to perform any intermediate preparation steps.

RPE cells can be produced, for example, according to the methods of Idelson M, Alper R, Obolensky A et al. (Directed differentiation of human embryonic stem cells into functional retinal pigment epithelium cells. Cell Stem Cell 2009; 5:396-408) or according to Parul Choudhary et al, (“Directing Differentiation of Pluripotent Stem Cells Toward Retinal Pigment Epithelium Lineage”, Stem Cells Translational Medicine, 2016), or WO 2008129554, all of which are incorporated herein by reference in their entirety.

The number of viable cells that may be administered to the subject are typically between at least about 50,000 and about 5×10⁶ per dose. In some embodiments, the RPE cell compositions comprise at least about 100,000 viable cells. In some embodiments, the RPE cell composition comprises at least about 150,000 viable cells. In some embodiments, the RPE cell composition comprises at least about 200,000 viable cells. In some embodiments, the RPE cell composition comprises at least about 250,000 viable cells. In some embodiments, the RPE cell composition comprises at least about 300,000 viable cells. In some embodiments, the RPE cell composition comprises at least about 350,000 viable cells. In some embodiments, the RPE cell composition comprises at least about 400,000 viable cells. In some embodiments, the RPE cell composition comprises at least about 450,000 viable cells. In some embodiments, the RPE cell therapy composition comprises at least about 500,000 viable cells. In some embodiments, the RPE cell composition comprises at least about 600,000, at least about 700,000, at least about 800,000, at least about 900,000, at least about 1,000,000, at least about 2,000,000, at least about 3,000,000, at least about 4,000,000, at least about 5,000,000 at least about 6,000,000, at least about 7,000,000, at least about 8,000,000, at least about 9,000,000, at least about 10,000,000, at least about 11,000,000, or at least about 12,000,000 viable cells.

In certain embodiments, the RPE cell therapy may be formulated at a cell concentration of between about 100,000 cells/ml to about 1,000,000 cells/ml. In certain embodiments, the RPE cell therapy may be formulated at a cell concentration of about 1,000,000 cells/ml, about 2,000,000 cells/ml, about 3,000,000 cells/ml, about 4,000,000 cells/ml, about 5,000,000 cells/ml, 6,000,000 cells/ml, 7,000,000 cells/ml, 8,000,000 cells/ml, about 9,000,000 cells/ml, about 10,000,000 cells/ml, about 11,000,000 cells/ml, about 12,000,000 cells/ml, 13,000,000 cells/ml, 14,000,000 cells/ml, 15,000,000 cells/ml, 16,000,000 cells/ml, about 17,000,000 cells/ml, about 18,000,000 cells/ml, about 19,000,000 cells/ml, or about 20,000,000 cells/ml.

In some embodiments, the RPE cell composition may be cryopreserved and stored at a temperature of between about −4° C. to about −200° C. In some embodiments, the RPE cell composition may be cryopreserved and stored at a temperature of between about −20° C. to about −200° C. In some embodiments, the RPE cell composition may be cryopreserved and stored at a temperature of between about −70° C. to about −196° C. In some embodiments, the temperature adequate for cryopreservation or a cryopreservation temperature, comprises a temperature of between about −4° C. to about −200° C., or a temperature of between about −20° C. to about −200° C., −70° C. to about −196° C.

In some embodiments, the cell composition is administered in the subretinal space. In other embodiments, the cell composition is injected.

In some embodiments, the cell composition is administered as a single dose treatment.

In some embodiments, the RPE cells are administered in a therapeutically or pharmaceutically acceptable carrier or biocompatible media. In some embodiments, the volume of the RPE formulation administered to the subject is between about 10 μl to about 50 μl, about 20 μl to about 70 μl, about 20 μl to about 100 μl, about 25 μl to about 100 μl, about 100 μl to about 150 μl, or about 10 μl to about 200 μl. In certain embodiments, two or more doses of between 10 μl and 200 μl of the RPE formulation can be administered. In certain embodiments, the volume of RPE formulation is administered to the subretinal space of a subject's eye. In certain embodiments, the subretinal delivery method can be transvitreal or suprachoroidal. In some embodiments, for some subjects, the incidents of ERM may be reduced using a transvitreal or suprachoroidal subretinal delivery method. In some embodiments, the volume of RPE formulation can be injected into the subject's eye.

Subjects which may be treated include primate (including humans), canine, feline, ungulate (e.g., equine, bovine, swine (e.g., pig)), avian, and other subjects. Humans and non-human animals having commercial importance (e.g., livestock and domesticated animals) are of particular interest. Exemplary mammals which may be treated include, canines; felines; equines; bovines; ovines; rodentia, etc. and primates, particularly humans. Non-human animal models, particularly mammals, e.g. primate, murine, lagomorpha, etc. may be used for experimental investigations.

The RPE cells generated as described herein may be transplanted to various target sites within a subject's eye or other locations (for example in the brain). In accordance with one embodiment, the transplantation of the RPE cells is to the subretinal space of the eye, which is the normal anatomical location of the RPE (between the photoreceptor outer segments and the choroid). In addition, dependent upon migratory ability and/or positive paracrine effects of the cells, transplantation into additional ocular compartments can be considered including but not limited to the vitreal space, inner or outer retina, the retinal periphery and within the choroids.

The transplantation may be performed by various techniques known in the art. Methods for performing RPE transplants are described in, for example, U.S. Pat. Nos. 5,962,027, 6,045,791, and 5,941,250 and in Eye Graefes Arch Clin Exp Opthalmol March 1997; 235(3):149-58; Biochem Biophys Res Commun Feb. 24, 2000; 268(3): 842-6; Opthalmic Surg February 1991; 22(2): 102-8. Methods for performing corneal transplants are described in, for example, U.S. Pat. No. 5,755,785, and in Eye 1995; 9 (Pt 6 Su):6-12; Curr Opin Opthalmol August 1992; 3 (4): 473-81; Ophthalmic Surg Lasers April 1998; 29 (4): 305-8; Ophthalmology April 2000; 107 (4): 719-24; and Jpn J Ophthalmol November-December 1999; 43(6): 502-8. If mainly paracrine effects are to be utilized, cells may also be delivered and maintained in the eye encapsulated within a semi-permeable container or biodegradable extracellular matrix, which will also decrease exposure of the cells to the host immune system (Neurotech USA CNTF delivery system; PNAS Mar. 7, 2006 vol. 103(10) 3896-3901).

In accordance with some embodiments, transplantation is performed via pars plana vitrectomy surgery followed by delivery of the cells through a small retinal opening into the sub-retinal space or by direct injection.

The subject may be administered corticosteroids prior to or concurrently with the administration of the RPE cells, such as prednisolone or methylprednisolone, Predforte. According to another embodiment, the subject is not administered corticosteroids prior to or concurrently with the administration of the RPE cells, such as prednisolone or methylprednisolone, Predforte.

Immunosuppressive drugs may be administered to the subject prior to, concurrently with and/or following treatment. The immunosuppressive drug may belong to the following classes: Glucocorticoids, Cytostatics (e.g. alkylating agent or antimetabolite), antibodies (polyclonal or monoclonal), drugs acting on immunophilins (e.g. cyclosporin, Tacrolimus or Sirolimus). Additional drugs include interferons, opioids, TNF binding proteins, mycophenolate and small biological agents. Examples of immunosuppressive drugs include: mesenchymal stem cells, anti-lymphocyte globulin (ALG) polyclonal antibody, anti-thymocyte globulin (ATG) polyclonal antibody, azathioprine, BAS 1L1×1MAB® (anti-I L-2Ra receptor antibody), cyclosporin (cyclosporin A), DACLIZUMAB® (anti-I L-2Ra receptor antibody), everolimus, mycophenolic acid, RITUX 1MAB® (anti-CD20 antibody), sirolimus, tacrolimus, Tacrolimus and or Mycophenolate mofetil.

Immunosuppressive drugs may be administered to the subject, for example, topically, intraocularly, intraretinally, or systemically. Immunosuppressive drugs may be administered in one or more of those methods at the same time or the delivery methods may be used in a staggered method.

Alternatively, the RTA RPE cell therapy composition may be administered without the use of immunosuppressive drugs.

Antibiotics may be administered to the subject prior to, concurrently with and/or following treatment. Examples of antibiotics include Oflox, Gentamicin, Chloramphenicol, Tobrex, Vigamox or any other topical antibiotic preparation authorized for ocular use.

In some embodiments, the cell composition does not cause inflammation after it is administered. In some embodiments, the inflammation may be characterized by the presence of cells associated with inflammation.

AMD is a progressive chronic disease of the central retina and a leading cause of vision loss worldwide. Most visual loss occurs in the late stages of the disease due to one of two processes: neovascular (“wet”) AMD and geographic atrophy (GA, “dry”). In GA, progressive atrophy of the retinal pigment epithelium, choriocapillaris, and photoreceptors occurs. The dry form of AMD is more common (85-90% of all cases), but may progress to the “wet” form, which, if left untreated, leads to rapid and severe vision loss.

The estimated prevalence of AMD is 1 in 2,000 people in the US and other developed countries. This prevalence is expected to increase together with the proportion of elderly in the general population. The risk factors for the disease include both environmental and genetic factors.

The pathogenesis of the disease involves abnormalities in four functionally interrelated tissues, i.e., retinal pigment epithelium (RPE), Bruch's membrane, choriocapillaries and photoreceptors. However, impairment of RPE cell function is an early and crucial event in the molecular pathways leading to clinically relevant AMD changes.

There is currently no approved treatment for dry-AMD. Prophylactic measures include vitamin/mineral supplements. These reduce the risk of developing wet AMD but do not affect the development of progression of geographic atrophy (GA).

Cell implantation can be used to slow down the progression of the disease, induce regeneration of the RPE and restore central vision.

Without RPE, photoreceptor cells become inoperable. Therefore, detection of GA using imaging techniques is carried out by identifying scotomas in the visual field. In the eyes of some subjects with GA, the disease can initially progress in a unique pattern that avoids the areas of the retina where visual acuity is the highest, such as the fovea. In these subjects, the fovea is only affected in the late stages of the disease.

Accordingly, using the methods described herein, the therapeutic effects of retinal disease therapies, such as cell therapies, can be measured. In one embodiment, the method comprises a quantitative structural assessment and a quantitative functional assessment of the eye of a subject with a treated retinal disease.

A non-limiting list of diseases for which the effects of treatment may be measured in accordance with the methods described comprises retinitis pigmentosa, lebers congenital amaurosis, hereditary or acquired macular degeneration, age related macular degeneration (AMD), geographic atrophy (GA), Best disease, retinal detachment, gyrate atrophy, choroideremia, pattern dystrophy as well as other dystrophies of the RPE, Stargardt disease, RPE and retinal damage due to damage caused by any one of photic, laser, inflammatory, infectious, radiation, neo vascular or traumatic injury. According to a particular embodiment, the disease is dry AMD. According to another embodiment, the disease is GA.

The FDA has accepted measurements of ocular structure as an endpoint in clinical trials for the evaluation of retinal disease therapies. In certain embodiments, measurements of ocular structure can be made using fundus autofluorescence (FAF) imaging. Fundus autofluorescence allows for a precise measurement of the atrophic area of an eye with a retinal disorder. In FAF imaging, the atrophic area appears hyperfluorescent (dark) surrounded by normal retinal tissue that has a mild hyperfluorescence. In a large percentage of subjects with GA, the atrophic area is surrounded by a rim of intense hyperfluorescence. This hyperfluorescence is associated with areas of apoptosis and cell death. According to embodiments of the disclosed method, measurements of the hyperfluorescence can be used to ascertain disease progression, particularly after treatment. The slowing or arrest of disease progression can be demonstrated by the shrinking or disappearance of the rim of intense hyperfluorescence that surrounds the atrophic area.

In certain embodiments, subjects having GA with an active lesion (i.e., atrophic area or scar), as evidenced by the presence of a hyperfluorescent rim around the periphery of the atrophic area after FAF imaging, can be treated using the implantation of hESC derived RPE, according to the method described in WO 2016/108219, for example, incorporated herein by reference in its entirety, or a similar method or a new method with reduced immunosuppression. To measure the effect of the treatment on disease progression, the lesion is first artificially divided into two halves by inserting a line, generated by the FAF imaging device, that crosses the lesion in parallel with the treatment area. The line is then moved perpendicularly towards the opposite side of the treatment area until the two parts of the lesion have a similar area. The position of the line across the lesion area of the retina is kept constant throughout the subjects' subsequent measurements. One half of the lesion area then receives treatment with implanted hESC derived RPE (the treatment area) and the other half of the lesion remains untreated.

At specified times after treatment, FAF can then be used to detect any hyperfluorescence, particularly around the rim of the lesion and the size of the area of atrophy can be measured. hi addition to the decrease in overall size of the lesion, a decrease in the size or disappearance of the hyperfluorescent rim around the periphery of the lesion can be used to indicate that the treatment is slowing down or arresting disease progression. The difference in hyperfluorescence between the treated half of the lesion and the nontreated half of the lesion can be measured and used to determine the efficacy of the treatment. As such, the same eye may be used as a treatment subject and control subject.

The determination that FAF can be utilized to demonstrate that a treatment area has undergone a change from hyperfluorescent to hypofluorescent, thereby indicating a slowing or arrest in disease progression, is an improvement of the current treatment effect assessment techniques using FAF. This improved procedure can be used as a surrogate for treatment effect in clinical trials.

hi one embodiment, FAF is carried out using BluePeak Blue Laser Autofluorescence (Heidelberg Engineering GmbH, Max-Jarecki-StraBe 8 69115 Heidelberg Germany). BluePeak is a non-invasive, scanning laser fundus imaging modality that reveals metabolic stress in the retina using lipofuscin as an indicator. BluePeak images can reveal RPE and photoreceptor cell malfunctions.

In another embodiment, treatment effect assessment using the two-dimensional imaging of fundus autofluorescence is augmented using optical coherence tomography (OCT). OCT can be used to generate three-dimensional high-resolution images and can provide important cross-sectional information for the structural assessment of retinal layers, particularly in subjects being treated for retinal diseases. Using OCT, profile images of the layers of the retina can be obtained before and after treatment for a retinal disorder has been administered. In healthy eyes, the individual layers of the retinal tissue can be seen as well-defined bands. Conversely, the characteristic defects caused by AMD or GA, for example, can be seen as a sharply demarcated region of degradation in the RPE and photoreceptor layers. In many eyes with GA, OCT images can show the wedge-shaped hyporeflective structures that can develop between the Brunch membrane and outer plexiform layer. Identification and monitoring of such structures can be useful in defining OCT boundaries of photoreceptor layers, which are important in clinical trials of therapies that aim to preserve the viability of the retinal layer in patients with AMD and GA.

By combining the segmentation of retinal layers in OCT with the metabolic mapping of fundus autofluorescence, morphologic alterations associated with functional change can be seen more clearly. Using specialized software, lesion areas seen in FAF images can be quantified and followed over time. Treatment effects, including areas of RPE regeneration that cover a lesion, can also be identified and recovery of RPE can be quantified by measuring the thickness of the retina.

At present, OCT may not always be standard for the assessment of retinal morphology in clinical trials. However, according to embodiments of the described method, when OCT is used in conjunction with other structural and functional assessment techniques, the measurement of the effect of treatments can be optimized and can result in shorter clinical trials that require fewer patients.

Another aspect of the methods described herein includes a functional assessment component for measuring the effect of treatments for retinal disease. There are several functional assessment techniques currently available including, low-luminance visual acuity, contrast sensitivity assessment, reading speed assessment, microperimetry, and quality of life assessments. In one embodiment, improved methods for the use of microperimetry are described.

Low-luminance visual acuity and contrast sensitivity measure the effect of luminance and contrast on overall visual function, but do not allow for more detailed assessment of function across specific areas of the retina. The specific location of GA or other retinal disease lesions in the macula or fovea can dictate visual outcomes. Thus, a high level of detail is important for functional assessments of vision in subjects with disorders such as GA.

In microperimetry, specific areas of the retina are stimulated with points of light, and the subject presses a button to acknowledge perception of the stimulus. In addition to identifying functional and nonfunctional areas, stimulus intensity can be varied to also identify the relative sensitivity of specific areas of the retina. The fundus can be monitored through an infrared camera and the sensitivity of the visual field can be mapped to the fundus photo and compared with images obtained with other techniques.

In some embodiments, healing of the injection site occurs within about 1 day (24 hours), 1 week, about 2 weeks, about 3 weeks, about 4 weeks, about 5 weeks after the treatment procedure. In other embodiments, healing of the injection site occurs within about 1 day to about 30 days after administration of the RPE cells. In still other embodiments, healing of the site of administration by a cannula is within 5 days to about 21 days or within about 7 days to about 15 days.

In some aspects, the BCVA of a subject treated with RPE cells described herein shows an increase in BCVA after about 1 day, about 1 week, about 2 weeks, about 3 weeks, about 1 month, about 2 months, about 3 months, about 4 months, about 5 months, about 6 months, about 7 months, about 8 months, about 9 months, about 10 months, about 11 months, when compared to age-matched, sex-matched control, a baseline of the subject or a fellow eye measurements. In some aspects, the BCVA of a subject treated with RPE cell compositions described herein shows an increase in BCVA from about 1 month to about 1 year after treatment with RPE cells, as compared to age-matched, sex-matched control, a baseline of the subject or a fellow eye measurement.

In some embodiments, the subretinal pigmentation in a subject treated with RPE cells described herein is stabilized for about 1 month to about 24 months after administration of treatment. In some embodiments, the subretinal pigmentation in a subject treated with RPE cells described herein is stabilized for about 2 months to about 12 months, about 3 months to about 11 months, about 1 month to about 6 months, about 4 months to about 18 months after administration of treatment.

In some embodiments, about 1 month to about 24 months after administration of RPE cells to the subject, the subretinal pigmentation is stabilized. In some embodiments, about 2 months to about 24 months after administration of RPE cells to the subject, the subretinal pigmentation is stabilized. In some embodiments, about 2 months to about 12 months, about 3 months to about 11 months, about 1 month to about 6 months, about 4 months to about 18 months after administration of RPE cells to the subject, the subretinal pigmentation is stabilized.

Subjects undergoing allogeneic cell transplantation procedures, such as those described herein, may develop an immune response towards these cells, thereby limiting their survival and functionality. Therefore, the subjects may receive systemic immunosuppression therapy (low dose of immunosuppression based on the prescribing information of the drug) before, and/or after administration of the RPE cells, consisting of the topical steroidal treatment as customary following vitrectomy and long-term systemic treatment.

In other embodiments, the subjects will receive one day to three months of immunosuppression. In other embodiments, the subjects will receive one day to three months of immunosuppression after administration of the RPE cell treatment. One method is to provide a course of Prednisolone or Dexamethasone drops 4-8 times daily, with gradual taper. Systemic (PO) tacrolimus 0.01 mg/kg daily (dose will be adjusted to reach blood concentration of 3-7 ng/mL), from up to two weeks before transplantation and continued up to 6-weeks post transplantation, by investigator discretion.

Systemic (PO) mycophenolate mofetil, up to 2 gr/per day, given from up to two weeks before transplantation and continued for one year post transplantation, may be used.

In one aspect, a method to increase the safety of a subject being treated for dry-AMD does not include the administration of immunosuppression agents. In other aspects, the incidence and frequency of treatment emergent adverse events is lower than when the subject is administered immunosuppression.

### EXAMPLES

Reference is now made to the following non-limiting examples, which together with the above descriptions illustrate some embodiments of the present disclosure.

Generally, the nomenclature used herein and the laboratory procedures utilized in the present disclosure include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8^{th} Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

The rationale for the biologic activity of RPE cells is that hESC-derived RPE cells can be safely transplanted into the subretinal space of patients with macular degenerative disease resulting from RPE cell degeneration, which will replace dead or dying RPE with functional RPE and result in biologic benefit including a reduction in the growth rate of areas of atrophy and an associated slowing or cessation in loss of vision. The transplant with functional RPE may result in; 1) re-establishment of a functional RPE layer, 2) preservation of existing photoreceptors, 3) create a microenvironment that is conducive to continued survival of existing cells and cellular function and/or structure, and 4) ultimately slow or, reverse disease progression thereby maintaining visual acuity.

RPE cell transplants as described herein, reduce, decrease or stop progression of GA and associated loss of visual function; maintain photoreceptor function in transplant area based on microperimetry and/or multifocal ERG; demonstrate improvement or restoration of areas of normal anatomical structure as determined by changes in the ellipsoid zone (EZ) in affected areas, RPE engraftment as evidenced by OCT, and improved retinal thickness. In addition, the RPE transplants maintain foveal area vision, improve BCVA, low luminance test and/or reading speed.

In certain subjects (e.g., patients), the GA lesion size can be from about 0.1 mm² to about 500 mm²; from about 0.5 mm² to about 30 mm²; from about 0.5 mm² to about 15 mm²; from about 0.1 mm² to about 10 mm²; from about 0.25 mm² to about 5 mm²; from about 5 mm² to about 50 mm²; from about 100 mm² to about 500 mm²; from about 2 mm² to about 25 mm². The GA lesion size may be measured by methods described herein or methods know in the art.

Exclusion criteria include, patient cannot undergo vitrectomy, or has a history of uveitis, diabetic retinopathy, CRVO, BVO, AION, optic atrophy, ongoing therapy for active treatment of wet AMD using anti-VEGF, end-stage glaucoma, diabetic retinopathy, vascular occlusions, uveitis, Coat's disease, glaucoma, is phakic, or has presence of moderate to severe ERM.

The RPE cell transplant, in one embodiment is administered as a single injection of 100-250K RPEs in, for example, a thaw and inject formulation. The RPE transplant may require repeated dosing to be determined. In another embodiment, the RPE transplant is administered as a single injection of 100-250K RPEs with no need for repeat dosing. In certain embodiments, administration comprises transvitreal, sub-retinal injection. In other embodiments, administration comprises transvitreal, sub-retinal injection.

### Example 1

### Clinical Protocol

The safety and tolerability of RPE cells described herein were evaluated in a dose-escalating Phase Ulla clinical study in patients with advanced dry AMD accompanied by GA. Patients in cohort 1 (patients 1, 2, and 3, ages 74-80 with a BCVA of 20/200 or less), received a target dose of 50,000 RPE cells in a volume of 100 μl. Patients in cohort 2, (patients 4, 5 and 6, ages 65 and 82, who also had a BCVA of 20/200 or less), received a target therapeutic dose of 200,000 RPE cells in a volume of 100 μl. Cohort 3, (patients 7, 8, and 9, who had a BCVA of 20/200 or less), received 100,000 cells in 50 μl. The RPE cells discussed herein were successfully administered and there were no serious adverse events. Retinal imaging data showed that the RPE cells that were administered engrafted in the patients and settled into a monolayer which is a characteristic of naturally occurring RPE. For at least the first patient, the RPE cells continued to be present after one year. The second patient showed similar results at the 6-month time point. Additional cohorts of subjects will use a higher dose of between 200,000 and 500,000 RPE cells.

The data from cohort 1 showed stable vision and FAF readings that indicate biological activity in patients that had completed 9 and 12 month time point readings. In addition, this initial data suggests that the RPE cells being transplanting into patients both engraft and survive for at least one year, and potentially longer. There are also some early signs of biological activity.

The study design includes a single center Phase Ulla study of patients with advanced dry form AMD and geographic atrophy (GA) divided into four cohorts: the first 3 cohorts, each consisting of 3 legally blind patients with best corrected visual acuity of 20/200 or less, received a single subretinal injection of RPE cells, using escalating dosages of 50×10³ cells for cohort 1 and 200×10³ cells for cohort 2 and cohort 3. The fourth cohort will include 9 patients with best corrected visual acuity of from between 20/64 to about 20/400, from between 20/70 to about 20/400 or about 20/64 or less, who will receive a single subretinal injection of between 200,000 and 500,000 RPE cells.

Following a vitrectomy, cells are delivered into the subretinal space in the macular area via a cannula through a small retinotomy. A total volume of up to between about 50-250 μl cell suspension is injected in areas at risk for GA expansion.

Along with the surgical procedure, patients may receive light immunosuppression and antibiotic treatment, comprising the following:

1. Topical steroidal and antibiotic treatment as customary following vitrectomy: A course of topical steroid therapy (Predforte drops 4-8 times daily, with gradual taper) and topical antibiotic drops (Oflox or equivalent 4 times daily) over the course of 6 weeks.
2. Systemic (PO) Tacrolimus 0.01 mg/kg daily (dose will be adjusted to reach blood concentration of 3-7 ng/ml), from a week before transplantation and continued until 6 weeks post transplantation.
3. Systemic (PO) Mycophenolate mofetil, total 2 gr/day, given from 2 weeks before transplantation and continued for one year post transplantation.

Protocol enhancements allow the reduction of the required duration of immunosuppression from 12 months to three months. This is significant for the patients. We plan to administer the RPE cells without immunosuppression and expect increased safety and the same or improved efficacy.

Patients are assessed at pre-scheduled intervals throughout the 12 months following the administration of the cells. Post study follow-up occurs at 15 months, and 2, 3, 4 and 5 years post-surgery.

Patient inclusion criteria includes the following factors: Age 55 and older; Diagnosis of dry (non-neovascular) age related macular degeneration in both eyes; Funduscopic findings of dry AMD with geographic atrophy in the macula, above 0.5 disc area (1.25 mm² and up to 17 mm²) in size in the study eye and above 0.5 disc area in the fellow eye; Best corrected central visual acuity equal or less than 20/200 in cohorts 1-3 and equal or less than 20/64 in cohort 4 in the study eye by ETDRS vision testing; Vision in the non-operated eye must be better than or equal to that in the operated eye; Patients with sufficiently good health to allow participation in all study-related procedures and complete the study (medical records); Ability to undergo a vitreoretinal surgical procedure under monitored anesthesia care; Normal blood counts, blood chemistry, coagulation and urinalysis; Negative for HIV, HBC, and HCV, negative for CMV IgM and EBV IgM; Patients with no current or history of malignancy (with the exception of successfully treated basal/squamous cell carcinoma of the skin) based on age matched screening exam (at discretion of the study physician); Patients allowed to discontinue taking aspirin, aspirin-containing products and any other coagulation-modifying drugs, 7 days prior to surgery; Willing to defer all future blood and tissue donation; Able to understand and willing to sign informed consent.

Patient exclusion criteria includes the following factors: Evidence of neovascular AMD by history, as well as by clinical exam, fluorescein angiography (FA), or ocular coherence tomography (OCT) at baseline in either eye; History or presence of diabetic retinopathy, vascular occlusions, uveitis, Coat's disease, glaucoma, cataract or media opacity preventing posterior pole visualization or any significant ocular disease other than AMD that has compromised or could compromise vision in the study eye and confound analysis of the primary outcome; History of retinal detachment repair in the study eye; Axial myopia greater than −6 diopters; Ocular surgery in the study eye in the past 3 months; History of cognitive impairments or dementia; Contraindication for systemic immunosuppression; History of any condition other than AMD associated with choroidal neovascularization in the study eye (e.g. pathologic myopia or presumed ocular histoplasmosis); Active or history for the following diseases: cancer, renal disease, diabetes, myocardial infraction in previous 12 months, immunodeficiency; Female; pregnancy or lactation; Current participation in another clinical study. Past participation (within 6 months) in any clinical study of a drug administered systemically or to the eye.

Efficacy may be measured by duration of graft survival and by the examination of the following of the rate of GA progression, retinal sensitivity in engrafted regions, extent and depth of central scotomata, and changes in visual acuity.

Adverse Event (AE) means any untoward medical occurrence, unintended disease or injury or any untoward clinical signs (including abnormal laboratory findings) in subjects, users, or other persons whether or not related to the Investigational medical treatment.

Serious Adverse Event (SAE) means An Adverse event that led to a death, injury or permanent impairment to a body structure or a body function, led to a serious deterioration in health of the subject, that either resulted in: a life-threatening illness or injury, or a permanent impairment of a body structure or a body function, or in-patient hospitalization or prolongation of an existing hospitalization or in medical or surgical intervention to prevent life threatening illness, led to fetal distress, fetal death or a congenital abnormality or birth defect.

In the study, no SAEs were reported that were treatment related.

In this example, the eye chosen for RPE administration is the eye with the worst visual function. The surgery can be performed by retro-bulbar or peri-bulbar anesthetic block accompanied by monitored intravenous sedation or by general anesthesia, at the discretion of the surgeon and in discussion with the patient. The eye undergoing surgery is prepped and draped in sterile fashion according to the institution protocol. After the placement of a lid speculum, a standard 3-port vitrectomy is performed. This may include the placement of a 23G infusion cannula and two 23G ports. After visual inspection of the infusion cannula in the vitreous cavity, the infusion line is opened to ensure that structure of the eye globe is maintained throughout the surgery. A careful core vitrectomy can then be performed with standard 23G instruments, followed by detachment of the posterior vitreous face. This will allow unobstructed access to the posterior pole.

In this example, RPE are introduced into the subretinal space at a predetermined site within the posterior pole, preferably penetrating the retina in an area that is still relatively preserved close to the border of GA. Blood vessels are avoided. The cells are delivered to the subretinal space via formation of a small bleb, with a volume of 50-150 μl.

The delivery system may be comprised of a 1 mL syringe that through a 10 cm extension tube which is connected to a Peregrine 25G/41G flexible retinal cannula.

Any cells that refluxed into the vitreal space can be removed and fluid-air exchange may be performed. Prior to removal of the infusion cannula, careful examination may be performed to ensure that no iatrogenic retinal tears or breaks were created. The infusion cannula may then be removed. Subconjunctival antibiotics and steroids may be administered. The eye may be covered with a patch and plastic shield. The surgical administration procedure may be recorded.

In this example, a low dose of 50,000 cells/50-150 μL or 50,000 cells/100 μL, medium dose of 200,000 cells/100 μL (or 100,000 cells/50 μL) and a high dose of 500,000 cells/50-100 μL was used. Dose selection was based on the safety of the maximal feasible dose tested in preclinical studies and the human equivalent dose calculated based on eye and bleb size.

Treatments provided herein include a suspension of therapeutic RPE cells that are delivered subretinally. They are highly purified, differentiated human pluripotent stem cells that are also “xeno-free,” meaning that no animal products are used at any point in the derivation and production process. (For example, see Idelson M, et al. 2009. “Directed differentiation of human embryonic stem cells into functional retinal pigment epithelium cells.” Cell Stem Cell Oct 2, 5(4):396-408 and Tannenbaum S E, et al. 2012. “Derivation of xeno-free and GMP-grade human embryonic stem cells-platforms for future clinical applications.” PLoS One. 7(6): e35325, both of which are herein incorporated by reference in their entirety).

RPE cells administered in a clinical-stage study targeting the major unmet medical need of dry-AMD. Age-related Macular Degeneration, or AMD, is the leading cause of blindness in people over the age of 60. The number of people suffering with dry-AMD is estimated to be nine times the number for wet AMD. However, there are currently no approved products for dry AMD.

### Example 2

### RPE Cell Growth and Survival in 2 Initial Subjects

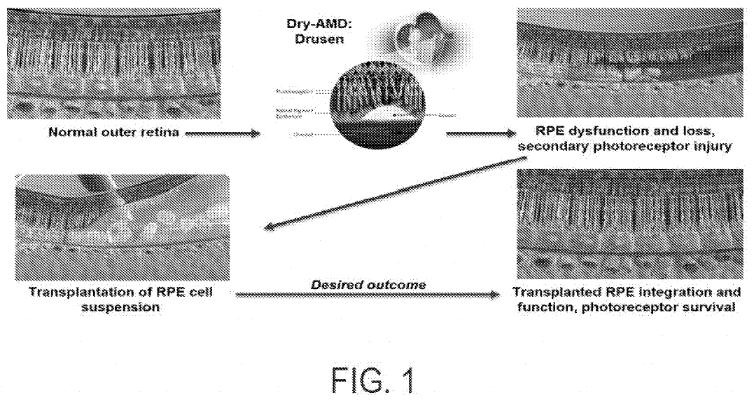

The effects of hESC derived RPE cell implantation to treat dry AMD and GA were measured in 2 initial subjects using embodiments of the methods described herein. The 2 subjects were treated with hESC derived RPE implantation according to the methods described in WO 2016/108219, or a similar method or a new method with reduced immunosuppression, as described above. New growth of RPE was demonstrated using OCT by measuring an increase in the thickness of the retina. Data indicating the implanted cells could survive for 6 months after transplantation under the retina was also collected. FIG. 1 shows a diagram of an example of cell-based therapy used to replace or support or both replace and support dysfunctional and degenerated RPE in dry AMD with GA.

The size of the lesion in these 2 initial subjects was measured using FAF. In addition, improved methods were used to measure the size of the hyperfluorescent rim around the periphery of the lesion to determine if the implanted cells had affected disease progression.

Data collected from these 2 subjects indicated that in the half of the lesion closest to the treatment area, hyperfluorescence had decreased or disappeared, demonstrating the cessation of disease progression.

### Example 3

### Safety and Efficacy Results for Cohorts 1 and 2 of the Clinical Study

Safety and imaging data from the patients in cohort 1 (patients 1, 2, and 3), who received a subretinal transplant of 50,000 RPE cells in suspension, and cohort 2 (patients 4, 5 and 6), who received a subretinal transplant of 200,000 RPE cells in suspension, are presented.

Patients were elderly, with significant loss of vision and large areas of clinically significant GA. The subject demographics and baseline characteristics are shown in Table 1.

TABLE 1
Subjects’ age and AMD characteristics at baseline.

| | Cohort 1 (n = 3) | Cohort 2 (n = 3) |
|---|---|---|
| Mean Age (in years) | 77.4 | 74.4 |
| Men: n | 1 | 0 |
| Women: n | 2 | 3 |
| Mean Visual Acuity in Treated Eye | ~20/800* | ~20/600 |
| Mean Area of GA | 13.95 mm² | 18.55 mm² |

*1 patient could only count fingers.

For cohorts 1 and 2, transplantation of RPE cells was performed by subretinal injection following a 23G vitrectomy under local anesthesia. Methods such as those described in WO 2016/108219, incorporated by reference herein in its entirety, may be followed, for example, to perform the injection. For the patients in cohorts 1 and 2, systemic immunosuppression was administered from 1 week prior to transplantation until 1 year after. However, methods which do not include immunosuppression may also be used. Systemic and ocular safety was closely monitored. Retinal function and structure were assessed using various techniques including BCVA, color and fundus autofluorescence (FAF) imaging, and OCT.

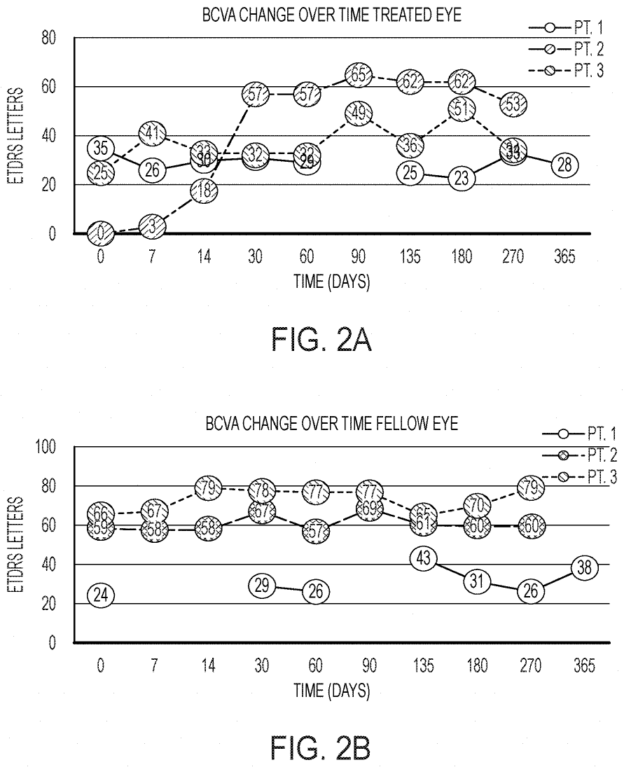

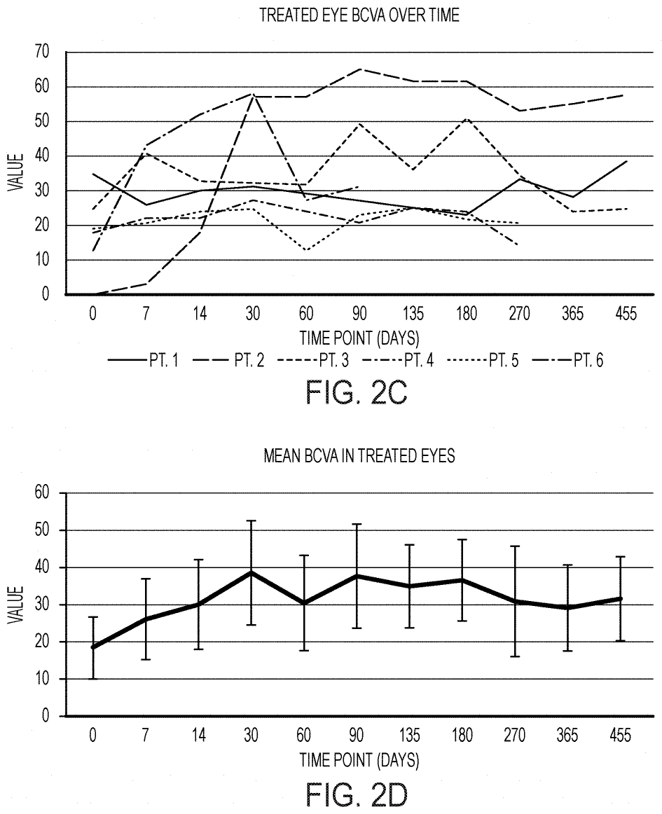

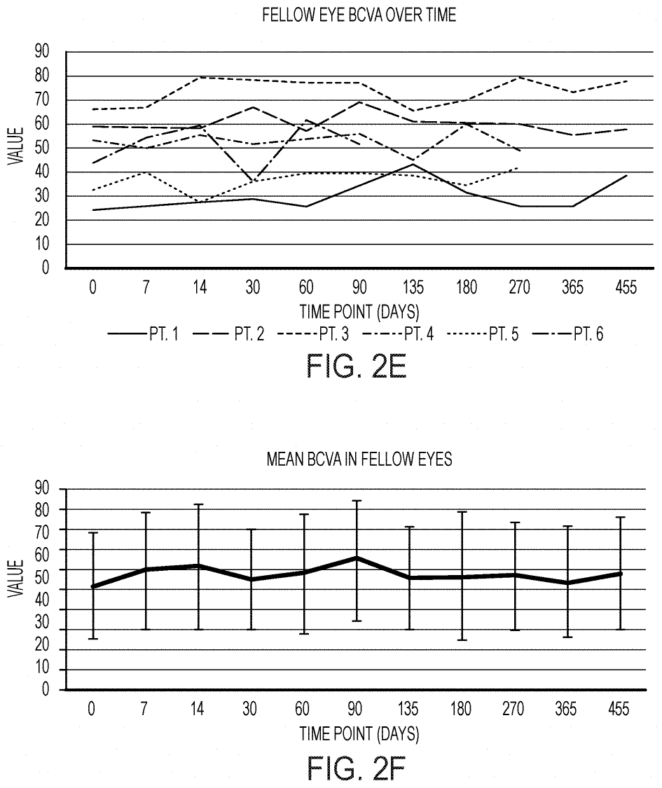

In FIG. 2A, the best corrected visual acuity (BCVA) is presented for the treated eye in cohort 1 (patients (Pt.) 1, 2, and 3). As shown, the BCVA did not decrease in the treated eye of patients 1, 2, or 3. Although patient 2 showed marked improvement, this may be partially associated with a clearing of vitreous and post capsule opacity that occurred during the surgery. The BCVA for the fellow eye is shown in FIG. 2B, which remained stable over the year in which it was tested.

The BCVA remained stable and did not decrease in the treated eyes of cohort 2 (patients 4, 5, and 6) and was stable in fellow eyes, as shown in FIG. 2C through FIG. 2F. Individual patients' treated eyes are shown in FIG. 2C and FIG. 2E.

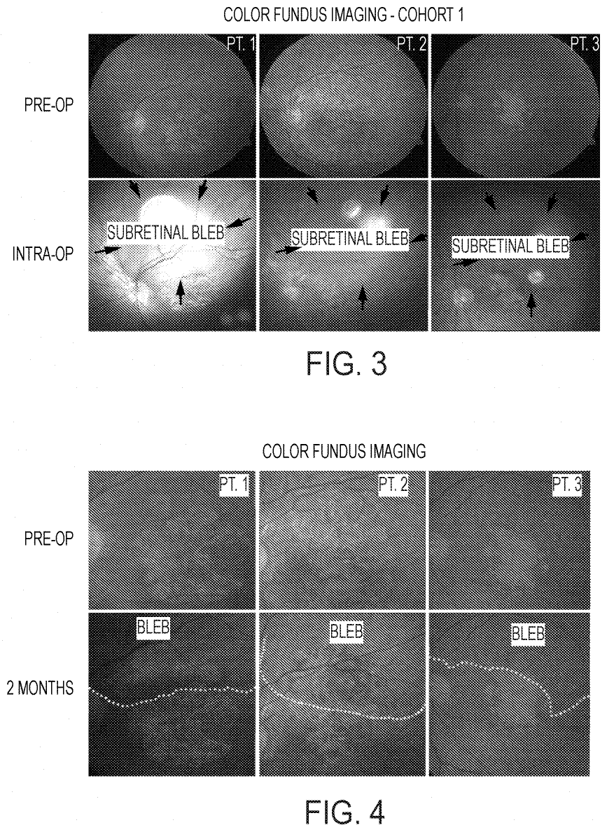

The retina comprises neurosensory tissue in the eyes that translates optical images into electrical impulses the brain understands. Fundus photography, which documents the retina, was also used to monitor the progression of the disease and treatment effects. Color fundus imaging for cohort 1 at prior to surgery (pre-op) and during surgery (intra-op) time points is shown in FIG. 3. The borders of the subretinal blebs (treatment areas) which occur following injection of the therapeutic RPE cell suspension are highlighted with arrows in the intra-op images. Surgery was uneventful, with subretinal fluid absorbing within less than 48 hours. As shown in FIG. 3, patients in cohort 1 have had large areas of GA develop and the images obtained intraoperatively demonstrate correct placement of transplanted cells.

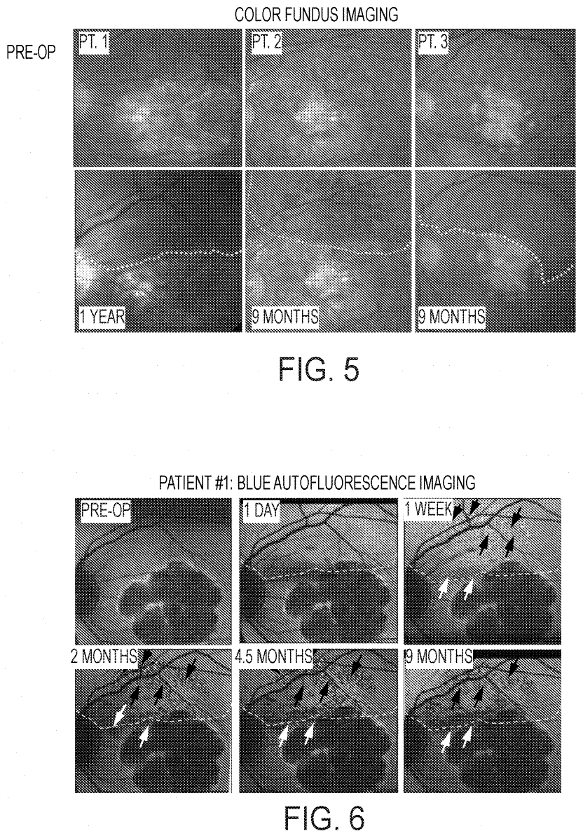

Color fundus imaging for cohort 1 at pre-op and 2-month time points is shown and compared in FIG. 4. Post-operatively, patients 1 and 2 show areas of subretinal pigmentation that developed in the inferior part of the subretinal bleb over the course of the first 2-3 months. After the first 2-3 months, subretinal pigmentation began to stabilize, as shown in FIG. 5.

Turning to FIG. 6, blue auto fluorescence images from patient 1 at pre-op, 1-day, 1-week, 2-month, 4.5-month, and 9-month post-op (following surgery) time points are provided. Blue fundus autofluorescence (FAF) imaging in a treated subject helps illustrate large areas of GA and the lower limit of the retina that was treated with RPE cells (outlined with dotted lines). These FAF images also indicate evidence of transplanted RPE cells as noted with black arrows, at the specified time points.

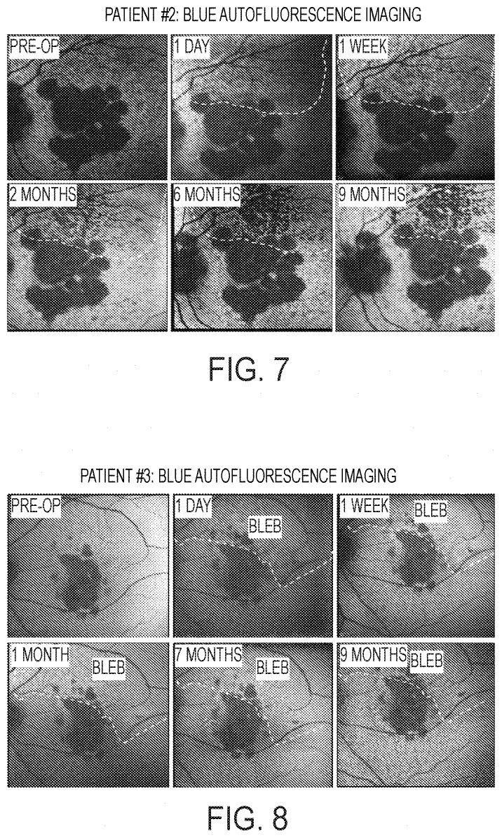

The blue auto fluorescence images from patient 2 at pre-op, 1-day, 1-week, 2-month, 6-month, and 9-month post-op time points can be seen in FIG. 7. The blue auto fluorescence images from patient 3 at pre-op, 1-day, 1-week, 2-month, 7-month, and 9-month post-op time points can be seen in FIG. 8.

Subretinal hypofluorescence and hyperfluorescence spots developed in the inferior area of the subretinal bleb in patients 1 and 2 over the course of the first 2-3 months, after which it stabilized. FIG. 6 and FIG. 7 demonstrate a progressive increase in cell number, pigment epithelium (PE) development and surface area covered by RPE cells, referenced by the black arrows in the upper right-hand corner of the post-op images of FIG. 6.

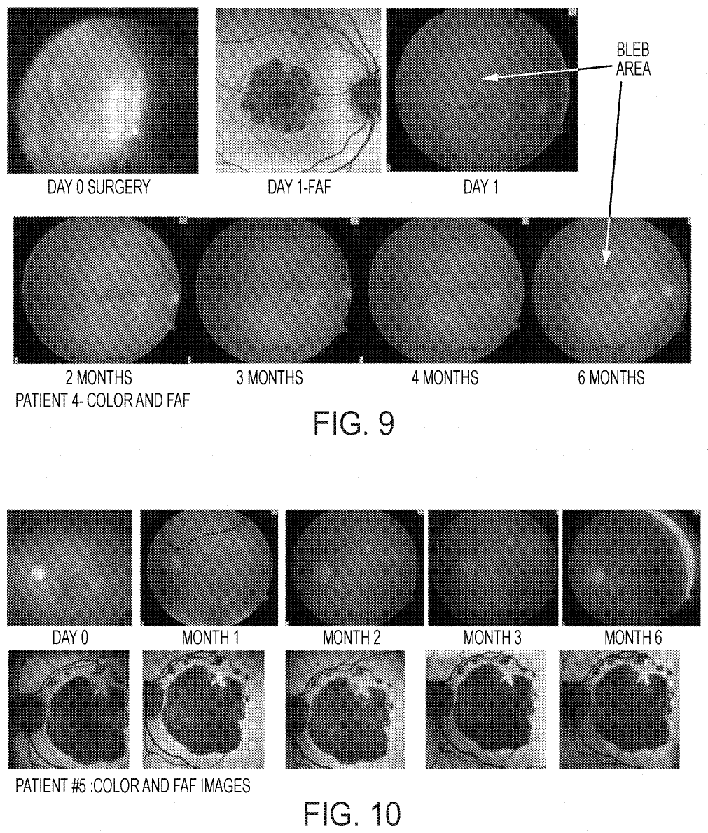

FIG. 9 shows a color image at the time of surgery (day 0), FAF and color images at day-1 post op, and color images at 2-months, 3-months, 4-months and 6-months post-op for patient 4 (cohort 2), which received a 200,000 RPE cell suspension dose. At the boundary of the bleb area, subretinal pigmentation can be seen up to 6 months. As shown in the images, gravity can cause the cells to settle and pigmentation to be localized at the bleb boundary.

FIG. 10 shows color and corresponding FAF images for patient 5 (cohort 2) at day 0, month 1, month 2, month 3, and month 6 post-op, who also received a 200,000 RPE cell suspension dose. As shown in FIG. 10, the treatment was well tolerated and stable pigment was increased by month 6.

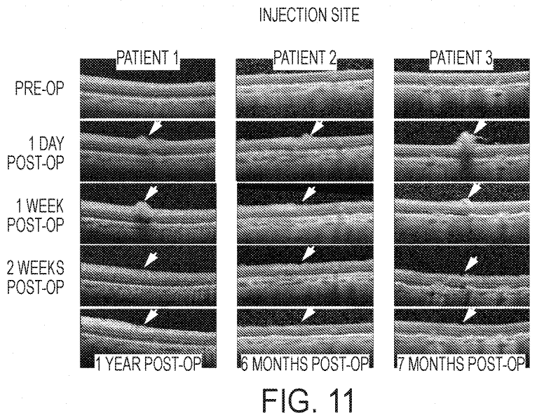

FIG. 11 shows healing of the injection site. As shown, subretinal fluid was absorbed rapidly (within less than 48 hours) and OCT images show healing of the site of retinal penetration by the cannula (arrows) within 2 weeks. A thin epiretinal membrane (ERM) developed in some cases.

OCT scanning can be used to analyze changes in the transition zone after treatment with RPE cells. In retinal degenerative diseases, a transition zone occurs between relatively normal retina containing healthy photoreceptors and severely affected retina with extreme photoreceptor atrophy (e.g., GA lesions, pre-GA lesions). Analysis of the transition zone for patients in cohort 1 (patients 1, 2, and 3) and cohort 2 (patients 4 and 5) using OCT scanning was performed.

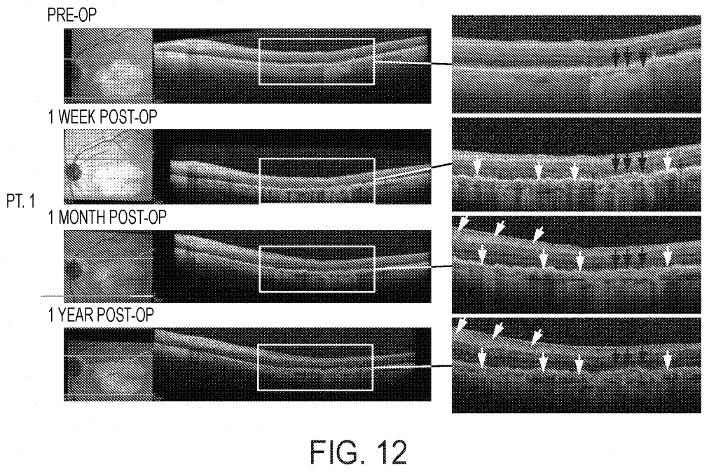

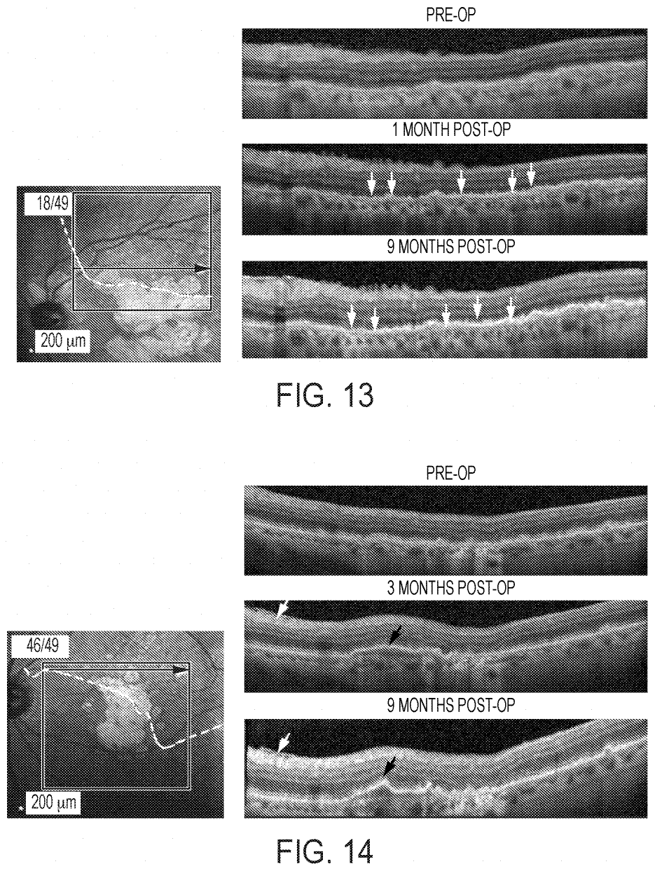

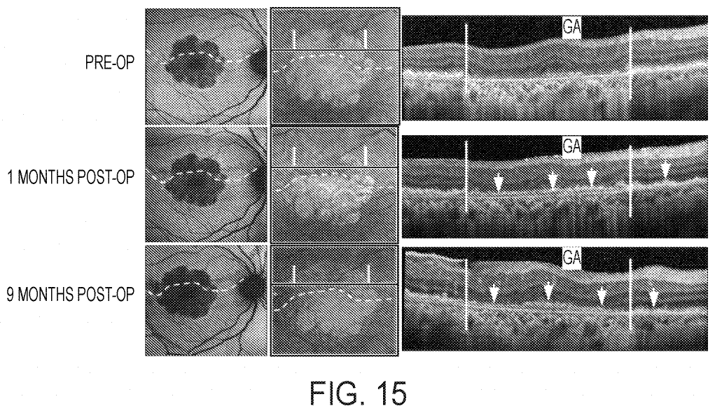

OCT scans were obtained for patient 1 at pre-op and 1-week, 1-month, and 1-year post time points, and are shown in FIG. 12. OCT scans for patient 2 are shown in FIG. 13 at pre-op and 1-month and 9-month post-op time points. FIG. 14 shows OCT scans for patient 3 of cohort 1 at pre-op, 3-month and 9-month post op-time points. FIG. 15 shows OCT and infrared OCT scans for patient 4 of cohort 2 at pre-op and 1-month post-op time points. FIG. 15 shows FAF (first column), infrared OCT scans (second column) and OCT scans (third column) for patient 4 of cohort 2 at pre-op and 1-month post-op time points.

The post-operative OCT scans in FIG. 12, FIG. 13 and FIG. 15 show irregular reflectance in the subretinal space of the treated area (yellow arrows), including regions which were atrophic at baseline (green arrows in FIG. 12). This irregular reflectance can indicate the presence of new RPE cells in the subretinal space. Images from a cohort 2 subject suggest subretinal layering of transplanted hESC-RPE cells. Images taken at baseline, one and 9 months follow-up with fundus autofluorescence (FAF), Infrared SLO OR SLO) and spectral domain OCT (SD-OCT) are presented. The white vertical lines show the limits of the geographic area in the IR SLO and OCT images. The green line represents the SD-OCT scan in the right column. The yellow dotted line represents the lower limit of the retina that was treated with RPE cells. This line was taken from the immediate post-op fundus picture and superimposed to the other image modalities.

Turning to the fundus images in FIG. 15, hypofluorescent spots can be seen in the lower portion of the treatment bleb over time, demonstrating a decrease in progression of the disease. Pigmentation can also be seen developing at the boundary of the bleb. In the infrared OC images (center column) in FIG. 15, pigmented cells can be seen obscuring the superior portion of GA (red lines indicate the boundary of GA) 1 month following surgery. This demonstrates that the cells have the ability to migrate and uniformly cover the upper portion of the GA and do not remain localized at the edge of the bleb. As infrared OCT has the ability to penetrate several layers of the retina, the cells, normal tissue, and the scar can all be observed.

In the last column of FIG. 15, the area of the GA can be seen denuded of RPE cells in the pre-op OCT image. However, OCT images taken at 1-month and 9-months post-op show RPE cells engrafted (yellow arrows). At 1-month, a uniform monolayer of RPE cells is shown covering the defect shown in the pre-op image, demonstrating a recovery of pigment epithelium and retinal thickness. At 9-months, the pigment epithelium is as thick as the normal cell area shown to the right and left of the GA boundary lines. Additionally, some areas demonstrated structural improvement in the ellipsoid zone (EZ). The EZ is an important area of the retina related to visual function where the RPE cells contact the photoreceptors and is the area of the retina where the visual process begins.

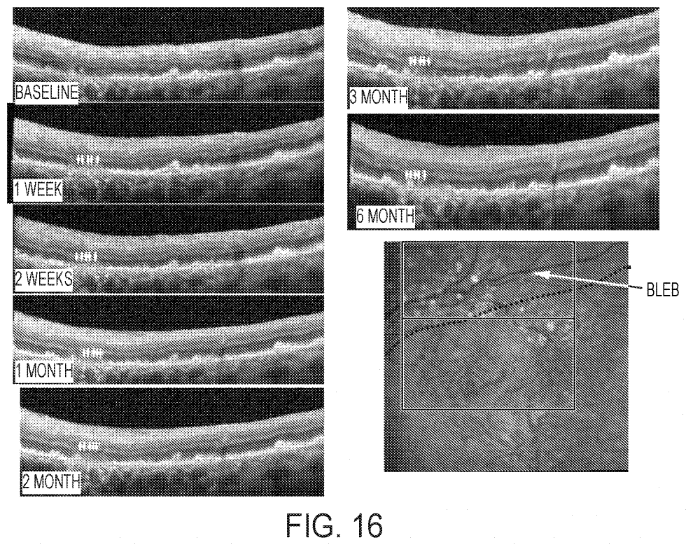

FIG. 16 shows OCT scans for patient 5 of cohort 2 (200,000 RPE cell suspension dose) at baseline, 1-week, 2-week, 1-month, 2-month, 3 month and 6-month post-op time points. The absence of observed edema or cysts (present when there is an autoimmune reaction) in patient 5 indicated that the treatment was well tolerated and that methods omitting immunosuppressants will produce comparable results.

Subretinal transplantation was well tolerated in all patients and accumulated data from cohorts 1 and 2, who received 50,000 or 200,000 cells in suspension with up to 15 months of follow up, showed no serious systemic and no unexpected ocular adverse effects. Following transplantation of hESC-derived RPE into the subretinal space of patients with advanced dry AMD, SD-OCT images show healing of the site of retinal penetration by the cannula within 2 weeks. BCVA remained stable, and subretinal pigmentation that correlates with irregular subretinal hyperreflectance in OCT imaging is evident in the majority of patients, demonstrating the presence of new RPE cells in the subretinal space. These results provide a framework for structural and functional assessments in future cohorts treated at higher doses of cells.

### Example 4

### Subretinal Transplantation of hESC-RPE Cells in a Porcine Eye

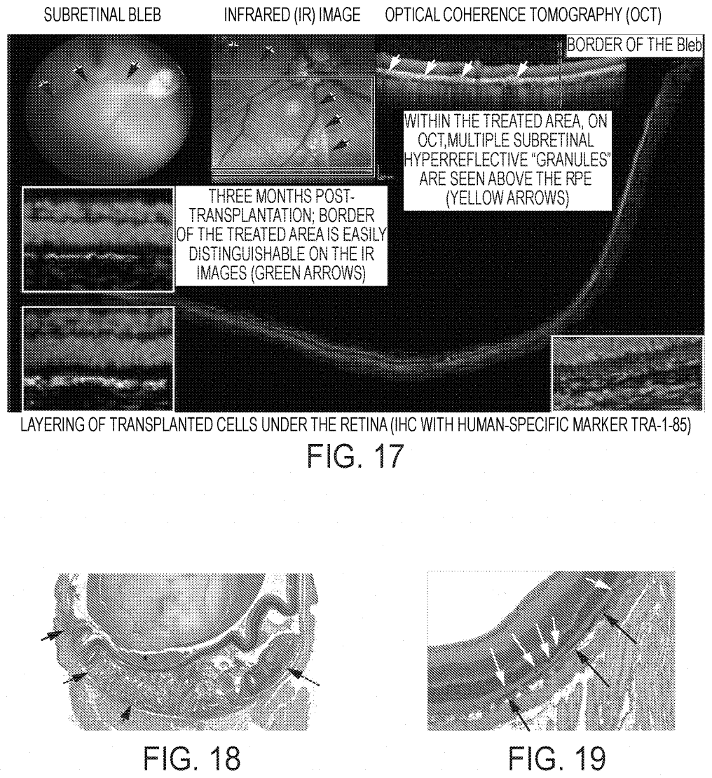

Human embryonic stem cell derived RPE cells (hESC-RPE cells) obtained by methods described above were transplanted subretinally into the eye of a pig to further analyze safety and cell survival. OCT scans were taken at 3-months post-operation (FIG. 17) and show irregular reflectivity in the subretinal space (yellow arrows in the upper right-hand image), similar to that seen in the treated patients of cohorts 1 and 2 (see FIG. 12 through FIG. 16). This irregular reflectivity can be compared to the area beyond the bleb border, where reflectivity of this layer is uniform (pink arrows).

Histological analysis was also performed. Immunohistochemistry (ICH) using the human-specific marker, TRA-1-85 was carried out. The TRA-1-85 antigen is a cell surface determinant expressed on almost all human cell types and is used in somatic cell hybrid studies to identify tissues of human origin. Upon histological examination, layering of the transplanted human cells under the retina was evident (shown in FIG. 17, in red). These results demonstrate that implanted RPE cells were present in the areas that showed irregular reflectivity on the OCT scans, months after administration, which could be distinguished from those native porcine RPE.

### Example 5

### Tumorigenicity, Engraftment and Survival of hESC-Derived RPE Cells in NOD-SCID Mice

Tumorigenicity, engraftment and survival of the hESC-derived RPE cells was tested in NOD-SCID mice for up to 9 months. In this assay, 100,000 hESC-derived RPE cells in suspension were injected into the subretinal space of NOD-SKID mice. The hESC-derived RPE cells were prepared according to the methods described above. The positive control group received hESC fragments, injected subretinally. The vehicle control group was injected with BSS Plus.

As shown in Table 2, no teratomas or human tumors were found in 142 mice injected subretinally with hESC-derived RPE at a dose of 100,000 cells. Surprisingly, there were no teratomas found in the group of mice injected subretinally with hESC-derived RPE, where the hESC-derived RPE cell suspension comprised up to 10% hESCs, which is 1,000 fold higher than would be injected into human subjects. Less than 5% of the mice had rare hESC-RPE proliferating cells found at 9 months. hi the mice injected with hESCs prepared similarly to the hESC-derived RPE cells at a dose of 100,000 cells in suspension, a suspension, a reduced potential for teratoma formation in the subretina (less than 15%) was demonstrated, as shown in Table 2. Teratomas were found in the majority (54.5%-80%) of the positive control animals injected with hESC fragments, as shown in FIG. 18 (arrows show the benign teratoma).

TABLE 2
Tumorgenicity and survival of hESCs, hESC fragments and
hESC-derived RPE at 9 months after being injected subretinally

| | % Mice w/ | | |
|---|---|---|---|
| | Histological | % Mice w/ | % Mice w/RPE |
| | Teratoma | Pigmented Cells | (HuNu⁺PMEL17⁺) |
| Single hESCs | 0%-15% | 0% | N/A |
| hESC Fragments | 54.5%-80% | 0% | N/A |
| hESC-derived RPE | 0% | 89.5%-96.4% | 83%-93% |

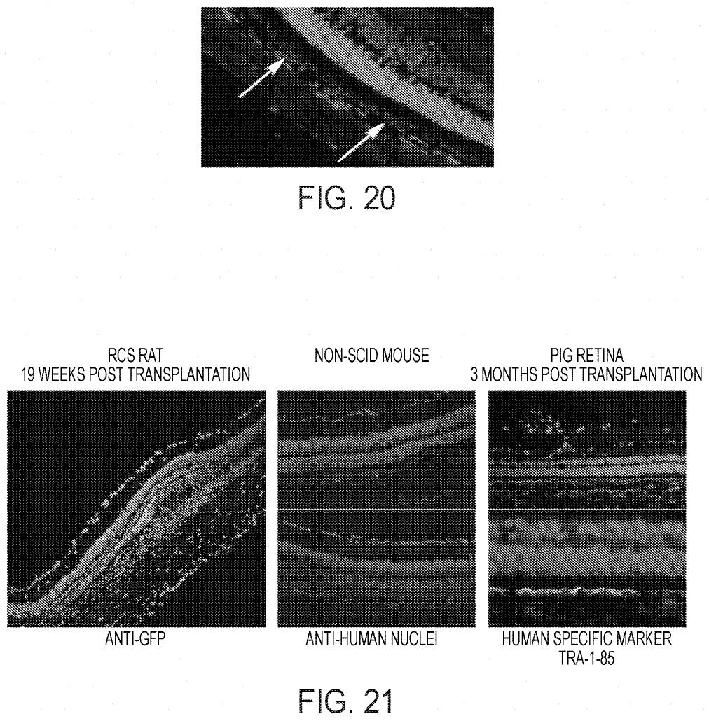

Long term consistent engraftment and survival was measured using histology in the subretrinal space after 9 months in those mice injected with hESC-derived RPE cells at a dose of 100,000 cells in suspension. As shown in Table 2, 89.5%-96.4% of the mice injected had pigmented cells and 83%-93% had RPE in the subretinal space. FIG. 19 shows the hESC-derived RPE in the subretinal space of mice injected with 100,000 hESC-derived RPE cells in suspension (arrows point to hESC-derived RPE in subretinal space). FIG. 20 shows an image of HuNu+PMEL17+ stained cells, demonstrating the presence of hESC-derived RPE cells in the subretinal space of mice injected with 100,000 hESC-derived RPE cells after 9 months. The human cell nuclei are stained with anti-human nuclei antibodies and mouse nuclei are counterstained with DAPI.

NOD-SCID mice (males and females) administered subretinally with a dose of up to 100,000 hESC-derived RPE demonstrated long term consistent hESC-derived RPE cell survival in the subretinal space and no product-related teratomas/tumors/abnormality over a 9 month study duration. Administration of hESC-derived RPE with up to 10% hESC impurity did not result in teratoma formation.

In addition, FIG. 21 shows the engraftment and survival of hESC-derived RPE in the retina of three animal species using stains that indicate the presence of human cells: RCS rat at 19 weeks post hESC-derived RPE transplantation, NON-SCID mouse at 9 months post hESC-derived RPE transplantation, and pig retina at 3 months post hESC-derived RPE transplantation. The arrows in the RCS rat retina image represent the location of anti-GFP staining and RPE cell engraftment, the arrows in the NON-SCID mouse retina image represent anti-human nuclei staining, and the arrows in the pig retina image represent staining of the human specific marker, TRA-1-85.

### Example 6

### Safety and Efficacy Results for Patient 8 of Cohort 3 of the Clinical Study

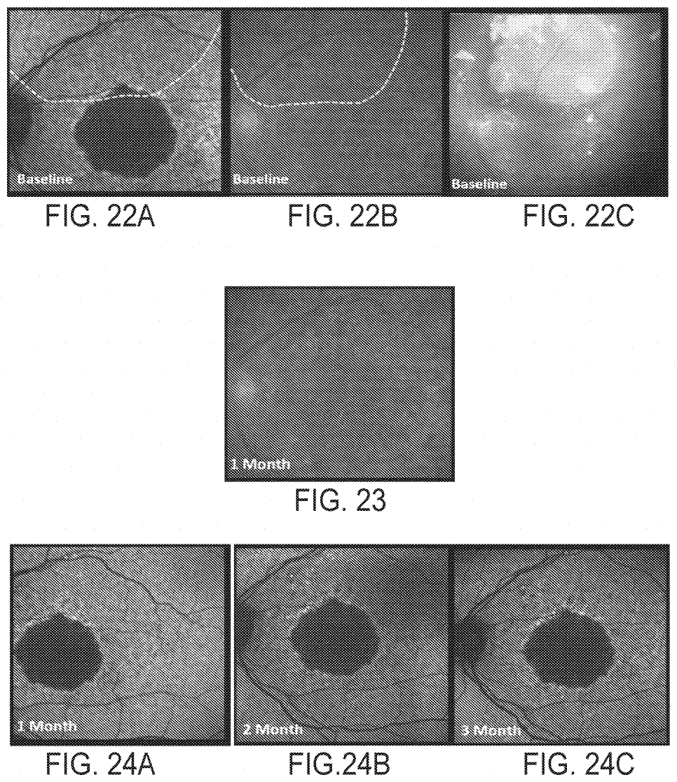

Patient 8 was administered 100,000 hESC-derived RPE cells in 50 μL subretinally, as described above. FIG. 22A is a blue auto fluorescence image taken before surgery, showing a baseline image of the GA (dark area), the outline of the future bleb border (dotted line) and the precise implantation location (star). FIG. 22B is a color fundus image taken before surgery, showing a baseline image of the GA (dark area), the outline of the future bleb border (dotted line) and the precise implantation location (star). FIG. 22C is a color image taken of the bleb implanted at the time of surgery.

FIG. 23 shows a color fundus image at 1 month. A slight subretinal hypofluorescence can be seen in the superior area of the bleb at 1 month.

FIG. 24A, FIG. 24B and FIG. 24C are blue auto fluorescence images taken at 1 month, 2 months, and 3 months, respectively. As shown in the images, hypofluorescent spots can be seen in the lower portion of the treatment bleb over time, demonstrating a decrease in progression of the disease. Pigmentation spots can also be seen developing within the bleb area.

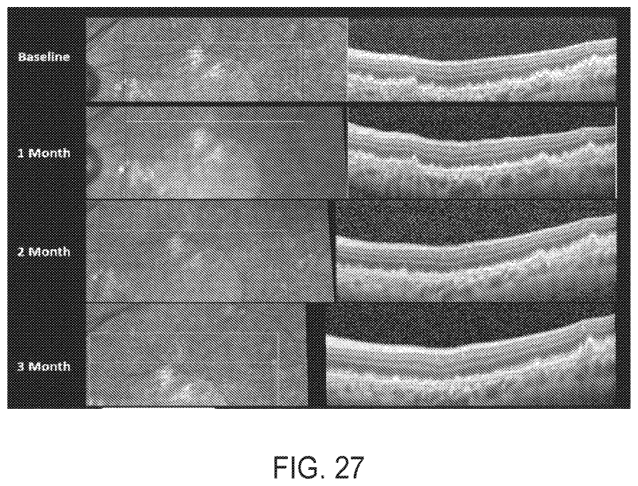

FIG. 25, FIG. 26 and FIG. 27 show infrared and corresponding OCT images at different cross-sections of the transition zone at time points of baseline (prior to surgery), 1 month, 2 months and 3 months for patient 8. The vertical arrows in the OCT images of FIG. 25 and FIG. 26 at the baseline and 1 month time timepoints show some of the drusen bodies present at these timepoints. A noticeable reduction in these drusen was observed at 2 months and 3 months after treatment with the hESC-derived RPE cell compositions. In addition, the OC images taken at the 3 month time point indicates a recovery and reestablishment of the ellipsoid zone, illustrated by the area highlighted by the horizontal arrows. These images indicate ellipsoid zone recovery according to an ellipsoid zone analysis. Ellipsoid zone analysis comprises, for example, a visual analysis of the ellipsoid zone. The ellipsoid zone analysis comprises a visual analysis of the ellipsoid zone, wherein the ellipsoid zone of a subject is compared to age-matched, sex-matched control, a baseline of the subject or a fellow eye of the subject.

Recovery is indicated, for example, by a subjective assessment of the inner segments and outer segments comprising the ellipsoid zone (EZ)—Inner segment and outer segment (IS/OS) junction. Recovery is indicated by a restoration of normal architecture (as shown in FIG. 25, FIG. 26 and FIG. 27 bottom image). Recovery, for example, is indicated by restoration of normal architecture as compared to age-matched, sex-matched control, a baseline of the subject or a fellow eye of the subject. Restoration of normal architecture indicates the potential restoration of vision. Recovery, for example, is shown by the subjective assessment that shows, for example, the beginnings of being able to see one or more of the external limiting membrane, myoid zone (inner segments of photoreceptors), ellipsoid zone (IS/OS Junction), outer segments of the photoreceptors, and loss of drusen. In some subjects there is a disappearance of reticular pseudo-drusen. In some embodiments, recovery is demonstrated by the organization of the basic foundational layers of the retina, organization of 2-6 of the 12-14 layers of the retina.

Recovery, for example, is the subjective assessment that one or more of the following are becoming more organized, including the, external limiting membrane, myoid zone (inner segments of photoreceptors), ellipsoid zone (IS/OS Junction), outer segments of the photoreceptors, loss of drusen, and disappearance of reticular pseudo-drusen. Recovery may also comprise the subjective assessment that one or more of the basic foundational layers of the retina are becoming more organized. As used herein, the basic foundational layers of the retina becoming more organized comprise one or more of the external limiting membrane, myoid zone (inner segments of photoreceptors), ellipsoid zone (IS/OS Junction), and outer segments of the photoreceptors.

The homogenous brownish color seen in the FAF images for cohorts 1-3 is consistent with pigmented cells in contrast to a blackish color seen when pigment dispersion occurs as a response after RPE injury. In at least 4 patients, pigmentary changes within the area of the bleb, both outside and inside the boundaries of the GA were seen. These changes in pigmentation, as well as areas of autofluorescence, seen in the FAF images correspond to the findings in the OCT images where new subretinal material can be seen as a fine layer resembling RPE in areas where patient RPE had disappeared. These results indicate that the implanted hESC-derived RPE cells have the ability to survive and graft to the host retina.

Assessment of surgical safety can include unhealing retinal detachment, proliferative vitreo-retinopathy (PVR), subretinal, retinal or intravitreal hemorrhage, and injury to relatively still healthy retina at the site of surgery. However, none of these events were observed in this study for any of the cohorts. The bleb formation did not cause disturbances to either the RPE or the neurosensory layers. Similarly, no retinal breaks or ruptures occurred. The lack of retinal breaks is noteworthy because retina covering a GA is thinner and the risk of causing retinal breaks is not negligible.

Findings using a variety of imaging modalities suggest the presence of cells in the subretinal space of human subjects, an observation supported by animal data in the mouse, rat and pig models studied using hESC-derived RPE cells. The surgical procedures were well-tolerated with SD-OCT images showing absorption of the subretinal fluid in the bleb within less than 48 hours after surgery and healing of the site of retinal penetration by the cannula within a few weeks. BCVA has remained stable in the treated eye of these advanced patients. Subretinal pigmentation that correlates with irregular subretinal hyperreflectance on OCT is evident in the majority of patients (5/6), suggesting the presence of cells in the subretinal space.

Future cohorts will have additional methods to actively assess visual changes and, based on these outcomes, will incorporate an additional variety of objective and subjective assessments such as microperimetry, low luminance visual acuity, reading speed, etc., to determine potential efficacy.

### Example 7

### Subretinal RPE Implantation Surgical Procedure

The surgical procedure is based on a conventional Pars Plana Vitrectomy (PPV) followed by subretinal injection of the cell suspension of RPE cells

Pre-Operative Phase

Pupil Dilation in the Operated Eye
Cyclopentolate Hydrochloride 1% (q 5 min×3)
Phenylephrine hydrochloride 2.5% (q 5 min×3)
Tropicamide 1% (q 5 min×3) or
as per surgical standard procedure of site

Anesthesia
Retro-Bulbar or subTenon block
General anesthesia may be performed per surgeon's criteria
Light sedation may be administered per surgeon's criteria
Peri or retrobulbar anesthetic agent given per standard of care (a commonly used combination consists of lidocaine 2% with bupivacaine 0.75%)

Cleaning
Povidone-iodine solution or as per surgical standard procedure of site

Vitrectomy
Perform a standard 3 port pars plana vitrectomy.
DORC is compatible with 23G.
   23G trocar system.
   Combination 23G/25G trocar system
   A 4th trocar for a “chandelier” type illumination may be added
Use of triamcinolone (ophthalmic) 40 mg/ml (4% concentration) to stain the vitreous and ensure complete separation of the posterior hyaloid:
   Undiluted triamcinolone acetonide (0.1 to 0.3 ml) is injected via a soft tip cannula into the vitreous cavity aiming towards the area to be visualized (e.g., optic disc and posterior pole)
Remove any vitreous traction (e.g., vitreomacular traction, significant epiretinal membrane) that is identified pre-operatively.
Optionally use intraoperative OCT (if available) to confirm that a full separation of the posterior vitreous face has been accomplished.

Preparation of Delivery Device (DD)
Carefully mix RPE cells 2-3 times by filling and discharging the syringe with the cell suspension into the vial
Load 0.35 mL RPE cells cell suspension into the syringe
Remove the 18G needle while holding the syringe upward, and release all air and air bubbles by pushing the plunger and gently tapping the syringe
Connect the syringe to the extension tube of the DORC delivery device
Fill the DORC extendible 41G subretinal injection needle with RPE cell suspension until a drop appears at the tip of the cannula
Slightly retract the plunger (or pump if using Microdose) to include a small amount of air in the tip (this will help recognize the tip is in the subretinal space during the initial air injection, help expand the subretinal space with air before the cell injection, and decrease the risk of cell reflux into the vitreous space during the cell injection)
Start the timer to record the time cells were kept within the device
The time from the prepared DD to implantation initiation should not exceed 2 minutes
Turn on the timer when the DD is ready and turn off when the implantation starts
Once the DD has been loaded and assembled, keep flipping/rotating the DD and do not leave flat/static as cells may settle out inside the syringe and tubing
Start cell implantation immediately and no later than 2 minutes after loading the DD
If more than 2 minutes have passed since the DD assembly, discard the loaded DD and prepare a new one

RPE Cell Implantation
Identify the area for injection that was previously selected based on patient images.
Area for injection should be at least 1-disc diameter away from the edge of the geographic atrophy (GA) lesion and located superiorly or superotemporally or over a GA lesion or over surrounding healthy tissue near a GA lesion.
Insert the cannula through the port and place tip at the pre-planned retinal location of injection; penetrate the retina carefully.
Slowly start injecting RPE cells into the subretinal space and verify that the tip of the cannula is in the subretinal space.
Once the bleb starts to form, slowly advance the tip of the cannula into the subretinal space (to avoid RPE cells from refluxing out of the subretinal space) and continue injecting slowly until the specified volume of RPE cells has been delivered to the subretinal space.
If the bleb seems to expand in undesired directions, cease injection and consider transplanting residual amount of RPE cells in a different location.

Should any reflux be noted during the implantation, the surgeon should immediately stop injecting RPE cells and proceed to perform a complete vitrectomy to make sure most refluxed cells in the vitreous were removed.

If no reflux is noted during the implantation, review the videotape before completing surgery to confirm no reflux occurred. If during the review reflux is noted, a complete vitrectomy should be performed to make sure most refluxed cells in the vitreous have been removed. In case an additional bleb is required (for the reason explained above), the location of the new bleb may be at or near the original bleb in which the RPE cells were implanted.
Make sure that the entire bleb is visible
Deliver 50 μL of the RPE cells cell suspension into the subretinal space slowly.
Gently and slowly withdraw the cannula.
Record time on the clock when surgery ended

Post-Surgery
At the end of the procedure, apply:
   SubTenon cefuroxime 0.1 cc (10 mg/ml) or equivalent antibiotics, and/or
   Maxitrol ophthalmic ointment (neomycin sulfate 3.5 mg in 1 g, polymyxin b sulfate 10000 [USP] in 1 g, dexamethasone 1 mg in 1 g), given once post-surgery,

Factors that could affect the outcome include, for example, the retinal area selected, the number of attempts to create a bleb (more attempts creates a less optimal outcome), any complications, the degree of reflux (none, mild, moderate, large), use of triamcinolone, cleaning of vitreous performed, if reflux occurred, if the pigmented cells in the vitreous were removed, and all concomitant medications given.
Eckardt, C., Tran's conjunctival suture less 23-gauge vitrectomy. Retina, 2005. 25(2): p. 208-11.
Fujii, G. Y., et al., A new 25-gauge instrument system for trans-conjunctival sutureless vitrectomy surgery. Ophthalmology, 2002. 109(10): p. 1807-12; discussion 1813.

TABLE 3
Summary of the Subjects 1-9

| | Procedure | | | | | |
|---|---|---|---|---|---|---|
| | | | | | Graft | Follow-up |
| | | | | | Survival | Period |
| Subject | HRA | OCT | FAF | CFP | (month) | (month) |
| 1 (102) | + | + | + | + | 15 | 15 |
| 2 (109) | + | + | + | + | 24 | 24 |
| 3 (110) | − | − | − | − | 0 | 24 |
| 4 (111) | + | + | + | + | 15 | 15 |
| 5 (113) | − | unk | +* | +* | 15 | 15 |
| 6 (117) | unk | unk | unk | unk | unk | 9 |
| 7 (402) | − | − | − | − | 0 | 3 |
| 8 (203) | − | + | + | +* | 3 | 3 |
| 9 (503) | − | − | − | − | 0 | 1 |

*vague
HRA = Heidelberg Retina Angiograph;
OCT = Optical coherence tomography;
FAF = Fundus autofluorescence;
CFP = Color fundus (retinal) photography

Subjects 1-9 demonstrate, no treatment-related systemic SAEs to date, but there were two unrelated SAEs occurred in 2 subjects; no unexpected ocular AEs have been observed; expected AEs included surgery-related conjunctival hemorrhages, worsening of cataracts and epiretinal membrane formation (ERM); new or worsening ERM have been observed (8/9); and no retinal edema, suggesting no immune response to RPE cells.

Subjects 1-8 demonstrate that at least 75% of subjects have RPE cells from between 2-24 months after administration. At the time this data was prepared it was too early to see signs of the cells in Subject 9.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

1. A pharmaceutical composition for treating or slowing the progression of a retinal disease or disorder comprising as an active substance about between about 50,000 and about 500,000 RPE cells.

2. A pharmaceutical composition for stabilizing the RPE of a subject with a retinal disease or disorder comprising as an active substance about between about 50,000 and about 500,000 RPE cells.

3. The pharmaceutical composition of claim 1, comprising from about 500 cells per μl to about 10,000 cells per μl.

4. The pharmaceutical composition of claim 1, wherein at least 95% of the RPE cells co-express premelanosome protein (PMEL17) and cellular retinaldehyde binding protein (CRALBP).

5. The pharmaceutical composition of claim 4, wherein the RPE cells further express one or more of RPE65, PERF, bestrophin 1, or tyrosinase.

6. The pharmaceutical composition of claim 1, wherein the RPE cells are generated by ex-vivo differentiation of human embryonic stem cells.

7. The pharmaceutical composition of claim 1, wherein the RPE cells are generated by:
(a) culturing human embryonic stem cells or induced pluripotent stem cells in a medium comprising nicotinamide so as to generate differentiating cells;
(b) culturing said differentiating cells in a medium comprising nicotinamide and activin A to generate cells which are further differentiated towards the RPE lineage; and
(c) culturing said cells which are further differentiated towards the RPE lineage in a medium comprising nicotinamide, wherein said medium is devoid of activin A.

8. The pharmaceutical composition of claim 7, wherein said embryonic stem cells or induced pluripotent stem cells are propagated in a medium comprising bFGF and TGFP under non-adherent conditions.

9. The pharmaceutical composition of claim 7, wherein the medium of (a) is substantially devoid of activin A.

10. The pharmaceutical composition of claim 2, comprising from about 500 cells per μl to about 10,000 cells per μl.

11. The pharmaceutical composition of claim 2, wherein at least 95% of the cells co-express premelanosome protein (PMEL17) and cellular retinaldehyde binding protein (CRALBP).

12. The pharmaceutical composition of claim 11, wherein the RPE cells further express one or more of RPE65, PEDF, bestrophin 1, or tyrosinase.

13. The pharmaceutical composition of claim 2, wherein the RPE cells are generated by ex-vivo differentiation of human embryonic stem cells.

14. The pharmaceutical composition of claim 2, wherein the RPE cells are generated by:
(a) culturing human embryonic stem cells or induced pluripotent stem cells in a medium comprising nicotinamide so as to generate differentiating cells;
(b) culturing said differentiating cells in a medium comprising nicotinamide and activin A to generate cells which are further differentiated towards the RPE lineage; and
(c) culturing said cells which are further differentiated towards the RPE lineage in a medium comprising nicotinamide, wherein said medium is devoid of activin A.

15. The pharmaceutical composition of claim 14, wherein said embryonic stem cells or induced pluripotent stem cells are propagated in a medium comprising bFGF and TGFP under non-adherent conditions.

16. The pharmaceutical composition of claim 14, wherein the medium of (a) is substantially devoid of activin A.
